(19) United States
(12) Patent Application Publication
(10) Pub. No.: US 2006/0166927 A1
(43) Pub. Date: Jul. 27, 2006

(54) Compounds that bind to the interferon-gamma, preparation method thereof and medicaments containing same

(76) Inventors: David Bonnaffe, Paris (FR); Andre Lubineau, Dourdan (FR); Jocelyne Alais, Bures sur Yvette (FR); Olivier Gavard, Bruxelles (BE); Anna Dilhas, Orisay (FR); Hugues Lortat-Jacob, Saint Ismier (FR)

Correspondence Address: FOLEY & LARDNER LLP, 1530 PAGE MILL ROAD, PALO ALTO, CA, 94304, US

(21) Appl. No.: 10/518,177

(22) PCT Filed: Jun. 18, 2003
(86) PCT No.: PCT/FR03/01860

§ 371 Date: Dec. 15, 2004

(30) Foreign Application Priority Data
Jun. 19, 2002 (FR) 02/07562

(51) Int. Cl. A61K 31/737; C08B 37/00

(52) U.S. Cl. 514/54; 536/53

(57) ABSTRACT

Compound capable of binding to gamma-interferon (γ-IFN), chosen from the molecules corresponding to formula (I) below:

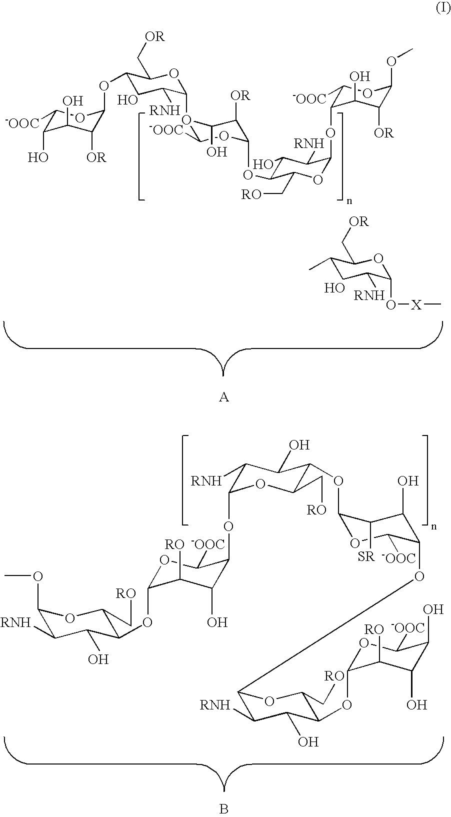

in which X is a divalent spacer group that is sufficiently long to allow the two oligosaccharide fragments A and B to each bind to one of the peptide sequences 125 to 143 of the C-terminal ends of a γ-interferon (γ-IFN) homodimer, n represents an integer from 0 to 10, and for example equal to 0, 1, 2, 3, 4 or 5, and each R independently represents a hydrogen atom, an SO₃⁻ group or a phosphate group, on the condition that no SO₃⁻ group is in the 3-position of the glucosamine units of compound (I). The invention also relates to the process for preparing these compounds, to the complexes formed by these compounds and gamma-interferon, and to the medicaments comprising these compounds or complexes.

The invention also relates to the process for preparing these compounds, to the complexes formed by these compounds and gamma-interferon, and to the medicaments containing these compounds or complexes.

Gamma-interferon (γ-IFN) is a polypeptide comprising, for example, 143 amino acids in humans, which is part of the cytokine family. Cytokines are mediators of cell communication which act according to a paracrine, autocrine or sometimes even endocrine process.

In the body, the production of these proteins is finely regulated and a deficiency or an excess in the synthesis thereof is generally responsible for various pathological conditions.

From a therapeutic point of view, it may therefore be advantageous to increase or, on the contrary, to reduce the biological activity of such a protein.

γ-IFN, first characterized on the basis of its antiviral activity, is involved in particular in controlling the immune response and during inflammation.

This cytokine is also cytotoxic or cytostatic for transformed cells and induces oxygenated radical synthesis. It regulates the expression of a large number of molecules of the pericellular space, in particular cell surface molecules, and also a large number of compounds of the extracellular matrix. γ-IFN therefore plays an important role, inter alia, in defence mechanisms, such as the immune response and inflammation, in cell growth and differentiation, and in adhesion and cell migration phenomena (1).

Therapies associated with cytokines, such as γ-IFN, consist either in administering this type of molecule or, on the contrary, in inhibiting the activities thereof.

Thus, the multiple activities mentioned above and observed in vitro have, themselves, given rise to many clinical trials in varied pathologies, such as cancer (2), chronic granulomatosis (3), rheumatoid arthritis (4), bacterial or parasitic infections (5), forms of hepatitis (6) or fibroproliferative diseases, such as systemic sclerosis (7). However, the clinical effectiveness of γ-IFN has not been clearly demonstrated to date, and its main indication remains limited to a rare disease: chronic granulomatosis (8).

Conversely, in certain inflammatory or autoimmune pathologies (9), or in order to decrease transplant rejection after transplantation, it may be advantageous to inhibit the biological activity of the cytokine. For this, antibodies or receptor in soluble form have been developed and tested on animal models (10).

The development of a therapy based on the use of γ-IFN poses considerable technical problems related in particular to its low half-life in vivo and to its poor bioavailability.

The obstacle provided by the poor bioavailability of γ-IFN can be overcome by using methods of local application, but these methods do not make it possible to reach the deep organs systemically; in addition, the problem of the short half-life in vivo remains untouched.

By way of example of such methods of local administration of γ-IFN, mention may be made of the inhalation of γ-IFN for the treatment of lung cancer, nebulization thereof in the treatment of the allergic response, or encapsulation thereof in liposomes.

A study of the cellular response to γ-IFN may make it possible to explain the difficulties encountered in therapeutic use.

In fact, the cellular response to γ-IFN depends on the type of cells stimulated, on the local concentration of γ-IFN and on the other regulatory factors to which the cell is concomitantly exposed.

In particular, it has been demonstrated that, independently of its cell receptor, γ-IFN is also capable of binding to oligosaccharides of the heparin or heparan sulphate (HS) type, with considerable affinity (5 to 10 nM) (11).

In vivo, in animals, the heparan sulphate effectively binds the γ-IFN, and this interaction controls the elimination of the cytokine from the plasma, its assimilation in various organs, and its location in the tissues.

In particular, after an intravenous injection, γ-IFN is eliminated by means of a biexponential process, during which 90% of the cytokine disappears from the circulating blood during the first 5 to 10 minutes, with a particularly short half-life time, in the region of 1 minute.

These results demonstrate that a large proportion, namely approximately 90% of the γ-IFN injected, is rapidly bound by molecules of heparin/heparan sulphate type, in particular at the surface of the vascular endothelium (12). In addition, autoradiographic observation of tissue sections shows that γ-IFN does not have identical access to all tissues.

It accumulates in the liver, the kidneys and the spleen, but not in the muscle, for example. In addition, within the same tissue, it is not evenly distributed and is concentrated in the heparan sulphate-rich zones. Such local concentrations are detected, for example, in the hepatic sinusoids—at the surface of the hepatocytes and of the endothelial cells—, in the renal glomerulae or in the red pulp of the spleen (13).

It therefore clearly appears that interaction of the cytokine with HSs considerably limits the accessibility to various compartments in vivo, and the difficulties encountered in the therapeutic use of γ-IFN (14) are probably related in part to these interactions. These studies also show that heparin sulphates are responsible for considerable local accumulations of cytokine in the tissues. Finally, it has also been shown that, in vivo, γ-IFN, alone, is rapidly inactivated by proteolytic degradation of its C-terminal end (12).

In order to provide a solution to the abovementioned problems, it has been proposed to associate γ-IFN with a heparin molecule. This association allows a much slower elimination from the plasma and a wider tissue distribution of the cytokine, and also induces an increase in activity by means of a mechanism of protection of the C-terminal domain against proteolytic degradations.

Moreover, injection of heparin alone makes it possible to displace the cytokine accumulated in the tissues by the endogenous HSs, and therefore to reduce or eliminate its activity.

However, the use of a heparin molecule for protecting γ-IFN and increasing its bioavailability or, conversely, for eliminating the local action thereof, raises difficulties which come from the activities of heparin, itself, and in particular from its anticoagulant properties.

The site of interaction for γ-IFN has been characterized on heparin sulphates (HSs). It consists of highly sulphated octasaccharide domains separated by a more extended 7 kD domain which is less sulphated.

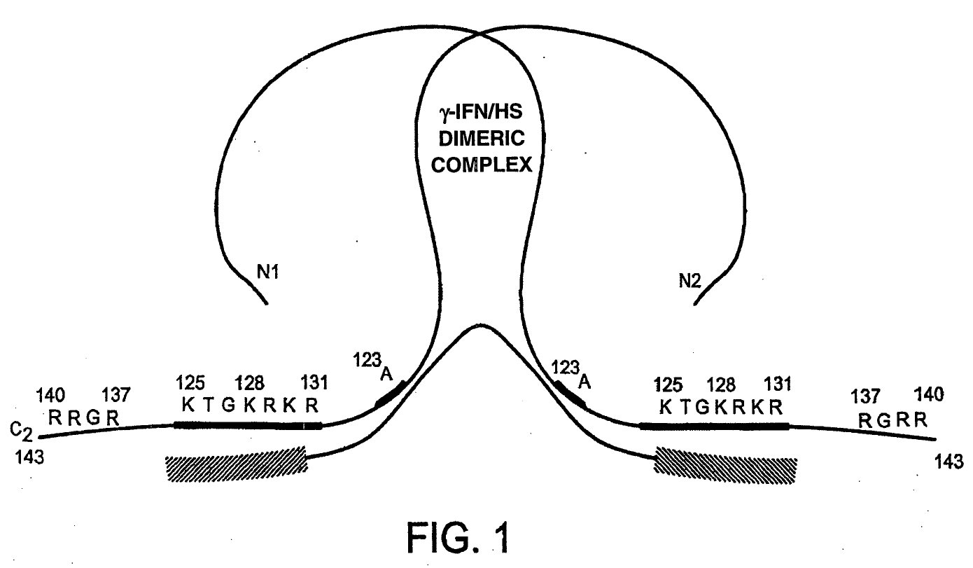

FIG. 1 shows a dimer complex of γ-IFN and of heparin sulphate. The two octasaccharide domains are represented by bold lines in FIG. 1.

Only the octasaccharide domains bind to the dimeric form of γ-IFN, the central domain forming a bridge between the two ends.

Document FR-A-2 736 832 (WO-A-97/03700) describes a γ-interferon activity-modulating agent comprising a group of formula A-X-B, in which A and B represent independently an oligosaccharide group carrying a sufficient anionic charge, for example in the form of sulphate groups, to confer on said oligosaccharide group an affinity for part of the C-terminal end of human γ-interferon containing the peptide sequence 125-131, and X is a spacer arm which is sufficiently long to allow the groups A and B to each bind to one of said peptide sequences of the C-terminal ends of a γ-interferon homodimer. The compound described in that document has the following drawbacks:

The molecules referred to as A and B are heparin or heparan sulphate depolymerization fragments. It may be recalled here that these molecules are characterized by a very great structural heterogeneity, and that no process exists for obtaining molecules of this type of defined structure, from natural sources. The compounds described in that document therefore in fact represent a mixture of molecules with varied structures. The same is true for the segment X which, depending on the case, may also be a heparan sulphate depolymerization fragment.

Such a molecule does not necessarily have C2 symmetry (the fragments A, X and B are “in line” or oriented in the same direction, or else are arranged in “parallel”) and does not therefore observe the symmetry of the protein to which it is supposed to bind.

Moreover, these molecules are of animal origin and have the drawback of transporting possible transmissible infectious agents.

There is therefore a need for a molecule which, when bound to γ-IFN, makes it possible, inter alia, to protect this molecule against any degradation, increases its bioavailability and also its half-life time, or is capable of displacing γ-IFN accumulated by heparan sulphates in the tissues.

In other words, there is a need for a molecule which, when associated with γ-IFN, has an action substantially similar to that of heparin, without having the drawbacks related thereto.

The aim of the invention is to provide a molecule capable of binding to γ-IFN, which satisfies, inter alia, the needs indicated above.

The aim of the invention is also to provide a molecule capable of binding to γ-IFN, which does not have the drawbacks, limitations, deficiencies and disadvantages of the similar molecules of the prior art, in particular heparin, and which solves the problems of the prior art.

This aim and others are achieved, in accordance with the invention, by providing a compound capable of binding to γ-interferon, γ-IFN, corresponding to formula (I) below:

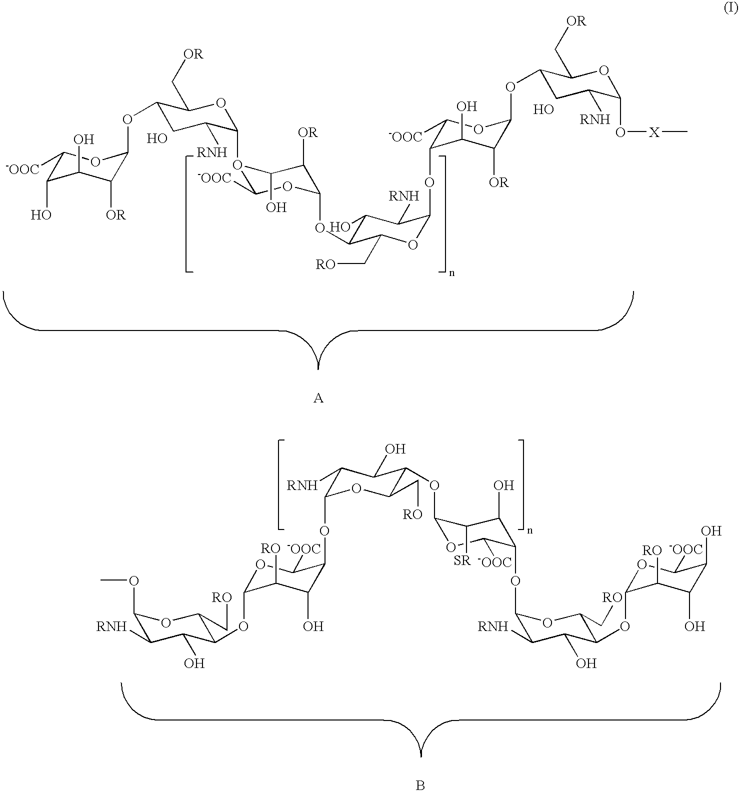

in which X is a divalent spacer group that is sufficiently long to allow the two oligosaccharide fragments A and B to each bind to one of the peptide sequences 125 to 143 of the C-terminal ends of a γ-interferon (γ-IFN) homodimer, n represents an integer from 0 to 10, and for example equal to 0, 1, 2, 3, 4 or 5, and each R independently represents a hydrogen atom, an SO₃⁻ group or a phosphate group, with the proviso that no SO₃⁻ group is in the 3-position of the glucosamine units of compound (I).

Preferably, all the R groups represent an SO₃⁻ group or all the R groups represent a phosphate group.

The molecule according to the invention is novel. It is not identical to any natural molecule nor to any of the molecules synthesized in the prior art, and in particular in application FR-A-2 736 832 (WO-A-97/03700).

In fact, the molecule according to the invention has a specific structure due, first of all, to the fact that the two oligosaccharide groups, placed on either side of the spacer group X, have an arrangement that can be described as “symmetrical” or “antiparallel” with respect to the spacer arm, whereas, both in natural heparin and heparan sulphate molecules and in synthetic heparins, and in the similar molecules described in the prior art, the two oligosaccharide groups have a “parallel” or “asymmetrical” arrangement. These molecules do not therefore observe the symmetry of the protein to which they are capable of binding.

In other words, the natural molecules and the molecules of the prior art, whether this involves heparin, heparan sulphate or molecules similar thereto, are completely and entirely asymmetrical, i.e. they are in a form of “1212” type, whereas the molecules of the invention are in an antiparallel form of type “1221”, i.e. having an C2 type symmetry.

Moreover, the molecules of the invention differ fundamentally from the natural molecules and from the molecules of the prior art by virtue of another essential structural characteristic, in the sense that the molecules according to the invention do not comprise a sulphate group in the 3-position of the glucosamine units.

Due to the fact that this sulphate group is predominantly responsible for the anticoagulant activity of heparin, the molecules according to the invention did not therefore have one of the main drawbacks of the molecules of the prior art, due to their anticoagulant activity.

The molecule according to the invention, which may be defined as being a “structural mimic” of heparan sulphate or of heparin, in which heparin-type oligosaccharides are linked by means of a hydrophilic spacer (X) of modulatable length, binds specifically to γ-IFN in the way that HSs do.

The compound according to the invention therefore has all the advantageous properties, and even more, of heparin: i.e., inter alia, the fact that it protects the γ-IFN molecule against attacks from proteases, an increase in bioavailability of the γ-IFN, the ability to dissociate, by competition, a γ-IFN/heparan sulphate complex, without having the essential drawback of heparin: i.e. the anticoagulant activity.

Finally, the molecules according to the invention are entirely synthetic molecules, unlike the molecules of the prior art, represented for example by document FR-A-2 736 832, in which the “end” oligosaccharide fragments are of natural origin, as is, in most cases, the spacer arm. The drawbacks exhibited by the molecules of document FR-A-2 736 832 have already been described above, in particular as regards the natural animal origin of the fragments which constitute them.

Due to their specific structure, the molecules according to the invention are therefore found to solve the problems of the molecules used for the same purposes in the prior art.

Advantageously, the spacer group is 15 to 150 Å, preferably 33 to 50 Å, in length.

The affinity for the cytokine is optimal for a length of 33 to 50, for example of 50 Å.

In general, the spacer group consists of a carbon chain, preferably of 1 to 120 C, in which one or more of the carbon atoms are optionally replaced with a hetero atom chosen from N, S, P and O, an SO₂ group, or an aryl group, said carbon chain also optionally carrying one or more anionic groups preferably chosen from sulphate groups, phosphate groups and carboxylic groups, etc.

Advantageously, and in particular due to the process used to synthesize the compounds according to the invention, the spacer group X is derived from a polyglycol preferably chosen from poly(alkylene glycols) in which the alkylene group comprises from 1 to 4 C, such as poly(ethylene glycol).

Thus, the spacer group may correspond to the formula:

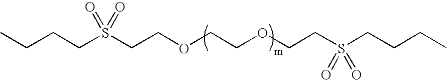

in which m is generally an integer from 5 to 32.

Furthermore, the compounds according to the invention will thus preferably correspond to formula (II) below:

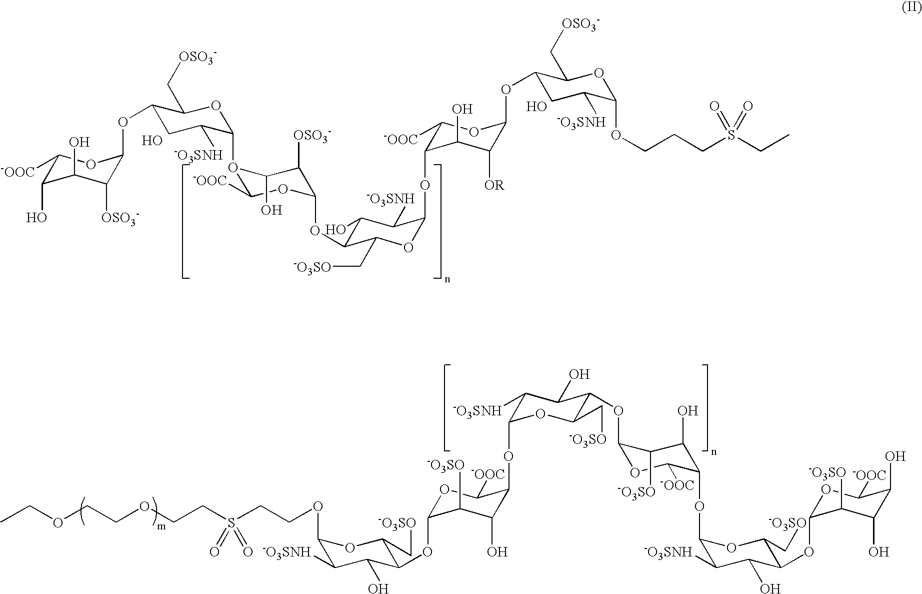

in which n and m have the meaning already given above.

The compounds of formula (II), that are particularly preferred are those in which n=0 and m=5 (IIa); n=0 and m=10 (IIb) ; n=0 and m=32 (IIc) ; n=1 and m=5 (IId); n=1 and m=10 (IIe); n=1 and m=32 (IIf); n=2 and m=5 (IIg) ; n=2 and m=10 (IIh); n=2 and m=32 (IIi).

The invention also relates to a process for preparing the compounds corresponding to formula (II), in which the free-radical coupling of two water-soluble compounds that are precursors of oligosaccharides of formula (III):

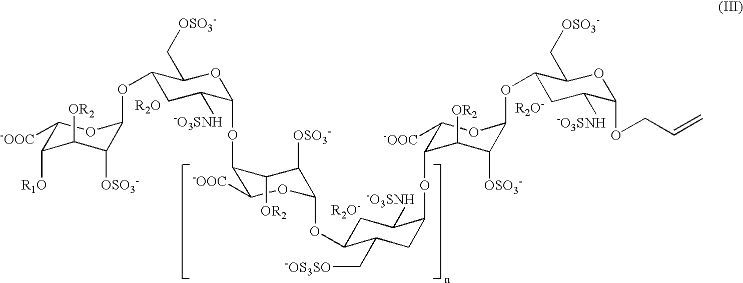

in which n is an integer from 0 to 10, for example equal to 0, 1, 2, 3, 4 or 5, and R₁ and R₂ represent a hydroxyl group-protecting group preferably chosen from p-methoxybenzyl and benzyl groups, with a dithiol compound that is a precursor of the spacer group of formula:

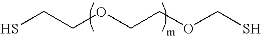

in which m is an integer from 5 to 32, is carried out so as to obtain a compound of formula (IV):

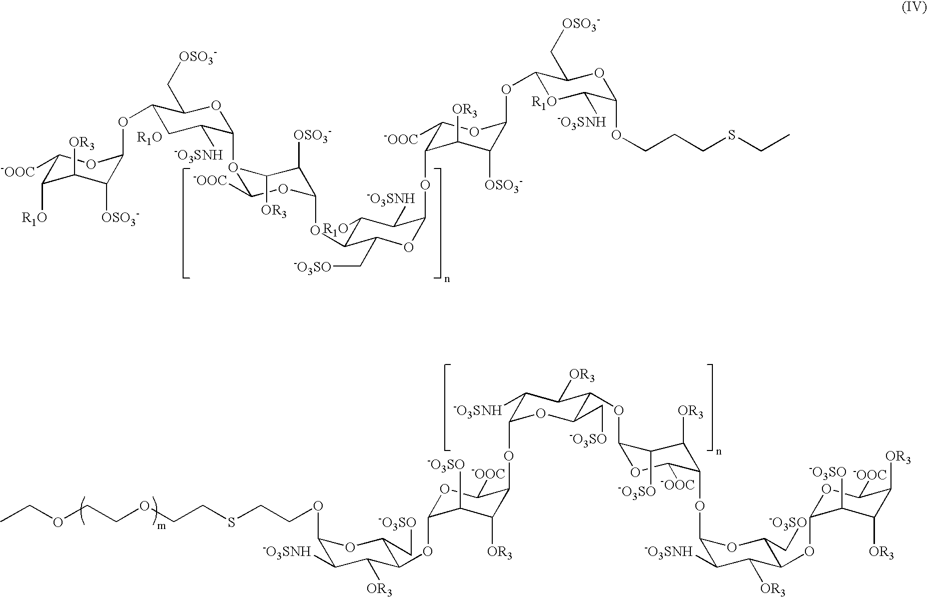

and then the thioether functions are oxidized to sulphones and the final deprotection of compound (IV) is carried out so as to give the final compound of formula (II):

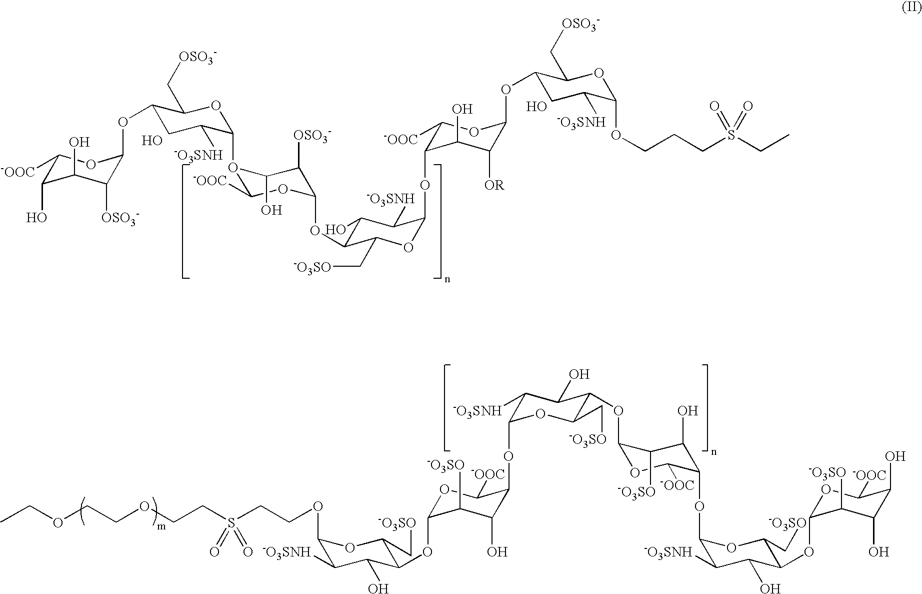

R₁ is preferably a p-methoxybenzyl group and R₂ is a benzyl group.

The water-soluble compound that is a precursor of oligosaccharides whose formula is given above (III) is prepared by means of the following successive steps:

a) a disaccharide of formula (V):

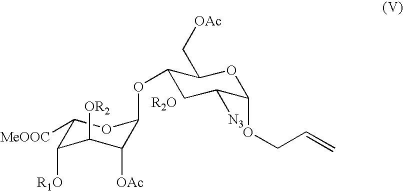

is subjected to oxidative cleavage of the R₁ group, preferably para-methoxybenzyl, so as to give an “acceptor” disaccharide of formula:

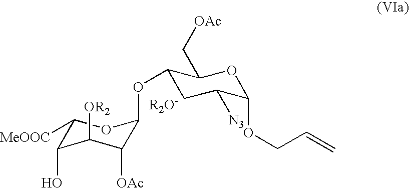

b) in parallel, a disaccharide of formula (V), above, is subjected to isomerization of the allyl group to 1-propenyl, followed by hydrolysis of the enol ether formed and activation of the hydroxyl group in the form of trichloroacetamidate, so as to give a “donor” disaccharide of formula (VIb):

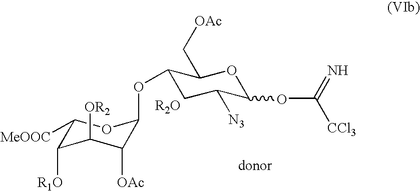

c) the acceptor disaccharide (VIa) and the donor disaccharide (VIb) are coupled so as to obtain the tetrasaccharide (n=0) of formula (VII), with an entirely alpha stereospecificity;

d) optionally, steps a) to c) are repeated, taking the tetrasaccharide prepared in c) as starting product for step a), so as to obtain the hexasaccharide (n=1) and octasaccharide (n=2) of formula (VII):

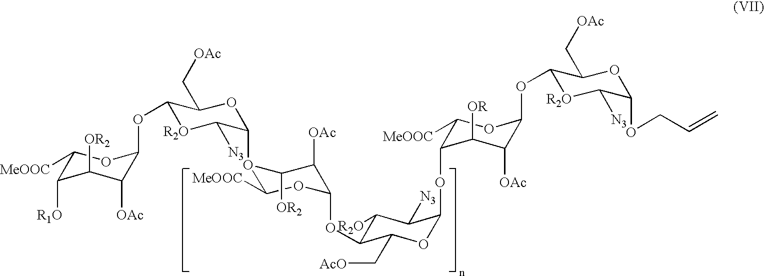

e) optionally, steps a) to c) are repeated, taking the octasaccharide prepared in d) as starting product for step a), so as to obtain a hexadecasaccharide (n=6) of formula (VII);

f) deacetylation, reduction of the azide function, sulphatation and saponification are carried out so as to obtain the desired water-soluble compound that is a precursor of the sought oligosaccharide (III).

The disaccharide of formula (V) is preferably prepared by means of a coupling reaction between a compound of formula (VIII):

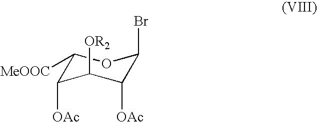

and a compound of formula (IX):

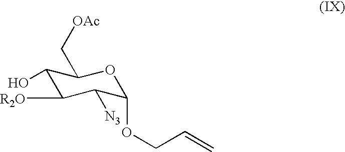

Finally, the compound of formula (IX) is prepared from the compound of formula (X) and the compound of formula (VIII) is prepared from the compound of formula (XI):

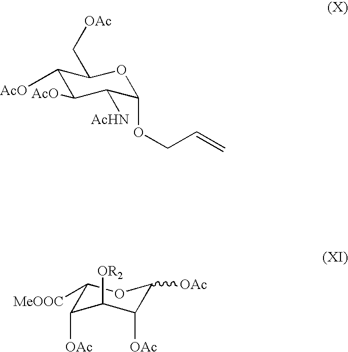

A preferred process for preparing compound (XI) is to acetylate the compound of formula:

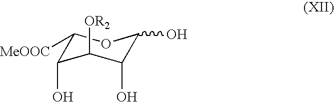

at −40° C. in dichloromethane as solvent, with pyridine as base, acetyl chloride as acylating agent, and 4-dimethylaminopyridine as catalyst.

The compound of formula (XI) is obtained with a very high yield, generally greater than or equal to 95%, and high purity, since the furano derivatives are only present in trace amounts, in an amount generally of about 2% or less.

A difficult purification is thus avoided according to the invention.

Throughout the following, the therapeutic uses of the compound according to the invention may use one but also several compounds according to the invention.

The invention also relates to compound (I) and to the preferred compounds (II), and (IIa) to (IIi), for use as a medicament, in general, compound (I) and compounds (II) and (IIa) to (IIi) being novel.

In other words, the invention relates to the use of compound (I) and of compounds (II) and (IIa) to (IIi), in general, for preparing a medicament.

When they are used alone, the compounds according to the invention can be used for modulating, for example inhibiting, the activity of exogenous or endogenous γ-interferon.

The invention therefore also relates to compound (I) and to compounds (II) and (IIa) to (IIi), for use as a modulator, for example inhibitor, i.e. reducer or suppressor, of the activity of endogenous or exogenous γ-interferon.

The invention also relates to compound (I) and to compounds (II) and (IIa) to (IIi) (alone: i.e. without any other active principle having a different structure), for use in the treatment of diseases associated with, or characterized by, the presence of pro-inflammatory cytokines such as γ-IFN; these are, for example, autoimmune, inflammatory, or degenerative diseases such as multiple sclerosis, glomerulonephritis, Crohn's disease and rheumatoid arthritis, transplant rejection, etc.

The invention thus also relates to compound (I) and to compounds (II) and (IIa) to (IIi) alone, for use in a treatment to supplement the immunosuppressive treatments used, for example, for preventing transplant rejection.

The invention also relates to the use of (I) (alone) and compounds (II) and (IIa) to (IIi), for preparing a medicament intended for the treatment of conditions or pathologies related to the activity, in particular excessive activity, of endogenous or exogenous γ-interferon, and to the use of (I) for preparing a medicament intended for the treatment of diseases associated with, or characterized by, the presence of pro-inflammatory cytokines such as γ-IFN; these are, for example, autoimmune, inflammatory or degenerative diseases such as multiple sclerosis, glomerulonephritis, Crohn's disease and rheumatoid arthritis, transplant rejection, etc.

The invention also relates to the use of a compound (I) and of compounds (II) and (IIa) to (IIi), for preparing a medicament intended for a treatment to supplement the immunosuppressive treatments used, for example, for preventing transplant rejection.

The invention further relates to a medicament containing a compound (or several compounds) of formula (I) or of formula (II) or (IIa) to (IIi), alone (i.e. without any other active compound); to a composition containing the compound (or several compounds) of formula (I), (II) or (IIa) to (IIi), alone, and a pharmaceutically acceptable vehicle, for use in the treatment of diseases associated with, or characterized by, the presence of pro-inflammatory cytokines such as γ-IFN (these are, for example, autoimmune, inflammatory or degenerative diseases such as multiple sclerosis, glomerulonephritis, Crohn's disease and rheumatoid arthritis, transplant rejection, etc.); or to a composition containing the compound (or several compounds) of formula (I), (II) or (IIa) to (IIi), alone and a pharmaceutically acceptable vehicle, for use in a treatment to supplement the immunosuppressive treatments used, for example, for preventing transplant rejection.

It should be recalled, in this respect, that gamma-interferon is a pro-inflammatory cytokine, the presence of which characterizes a certain number of pathologies associated with inflammation. In such situations, it is useful to suppress or reduce the biological activity of the endogenous gamma-interferon. In animals, experimental models have proved the advantage of such a strategy (inhibition of gamma-interferon) by using inhibitory monoclonal antibodies or a soluble form of the cytokine receptor. By way of example, mention may be made of autoimmune or degenerative diseases (multiple sclerosis, glomerulonephritis, Crohn's disease, rheumatoid arthritis, etc.). Similarly, the inhibition of gamma-interferon may be an effective supplement to immunosuppressive treatments, for example, with cyclosporine that are used, for example, to prevent transplant rejection.

The medicaments containing the compound (or compounds) (I) alone can be administered at doses which can be determined beforehand by means of routine experiments, according in particular to the desired effect. These doses may range, for example, from 0.1 to 200 mg per individual and per day, preferably from 1 to 50 mg.

The invention also relates to a medicament containing γ-interferon in addition to compound (I) or the preferred compounds (II), from (IIa) to (IIi).

Such a medicament contains a combination of compound (I) and γ-interferon, preferably in a proportion of 0.05 to 1 mg of γ-interferon and of 1 to 50 equivalents of compound (I).

In this medicament, compound (I) (for example (II) or (IIa) to (IIi)) and the γ-interferon are preferably in the form of a complex of compound (I) and of γ-interferon. Said complex makes it possible to increase the bioavailability of the cytokine and protects it against proteolytic degradations.

In other words, compound (I) prevents the capture of the γ-interferon by endogenous heparan sulphate molecules present, for example, in the extracellular matrix and at the surface of many cells, and therefore allows it to be maintained and transported in the general circulation.

In addition, compound (I) protects the γ-interferon against degradations which may reduce or eliminate its activity, and it makes it possible to maintain the γ-interferon in its most active form, up until the moment that it acts on competent cells.

The invention therefore relates to:
the complex of compound (I) (or (IIa) to (IIi)) and of γ-interferon, for use as a medicament. In fact, this complex is novel and its therapeutic use has never been mentioned;
the complex above, for use as an immunostimulant.

The immunostimulant effects comprise, for example, an antiproliferative effect in cancers and activation of the immune defences in infectious, for example viral, bacterial or parasitic, diseases, or else the ability to block collagen synthesis in organ fibroses, etc.

In addition, the invention will therefore relate to the complex above, for use in the treatment of a disease chosen from cancer, infectious, for example viral, bacterial or parasitic, diseases, and organ fibroses.

In general, the invention relates to a medicament containing said complex, and also to the use of the complex for the treatment of a disease as mentioned above.

The invention also relates to the use of the complex, in general, for preparing a medicament and, in particular, to the use of the complex for preparing a medicament intended for the treatment of a disease as mentioned above.

The invention also relates to a composition containing said complex and a pharmaceutically acceptable vehicle, for use in the treatment of a disease chosen from cancer, infectious, for example viral, bacterial or parasitic, diseases, and organ fibroses.

The invention will now be described in detail in the following description of a particular embodiment of the invention, in which the spacer group consists essentially of a polyglycol, in particular of a polyethylene glycol.

This description is given in relation to the attached drawings in which:

FIG. 1 represents a γIFN/HS or molecule according to the invention dimer complex;

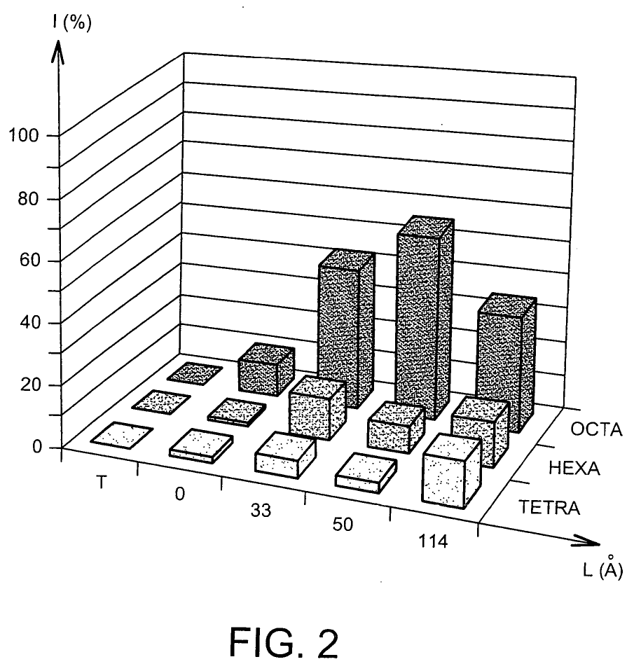

FIG. 2 is a graph which gives the % inhibition I for various molecules according to the invention, defined by the length L of the spacer arm (in Å) and the number of saccharides of the oligosaccharide groups (tetra-, hexa- or octasaccharide).

The compounds according to the invention can be defined as structural mimics of heparin or of heparan sulphate or else as neoglycoconjugates corresponding, in particular, to formula (II).

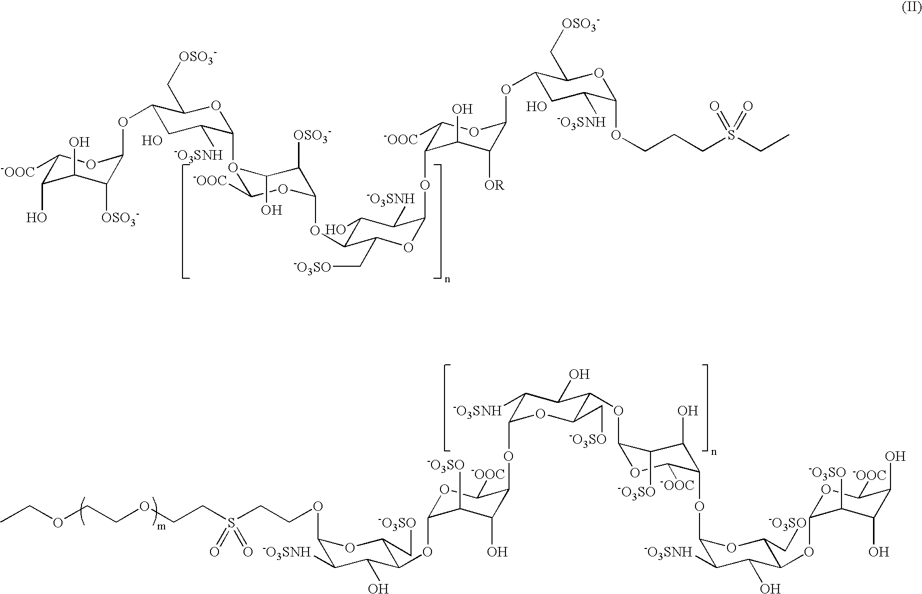

In these structural mimics, tetra- to octasaccharides, of heparin type, are linked by means of a hydrophilic spacer of modulatable length, for example a spacer of polyglycol type.

The molecules according to the invention bind specifically to γ-IFN, in the way that heparan sulphate does, as is described in FIG. 1.

The development of a process for preparing the compounds according to the invention essentially came up against four problems:
1. Obtaining high-yield coupling between the spacer and the oligosaccharides, which always requires a substantial amount of synthesis.
2. Controlling the stereochemistry of the bonding between the spacer and the first glucosamine unit of the oligosaccharide. This stereochemistry should be alpha, since it is the stereochemistry that is naturally found in the heparan sulphate (HS) polymer.
3. Controlling the alpha-stereochemistry of all the glycoside bonds in the oligosaccharide.
4. Obtaining effective access to L-iduronic acid, which is a rare sugar that cannot be isolated from natural sources in sufficient amounts for synthetic needs.

In the following text, we describe the synthesis of the compounds according to the invention, each time emphasizing the solutions provided, by the process of the invention, to each of the problems of synthesis listed above.

1. Development of Efficient Coupling to a poly-(ethylene glycol) (PEG) Spacer

For the coupling between the oligosaccharides and the spacer, use is preferably made, according to the invention, of a conventional organic chemistry reaction: free-radical coupling of a thiol to an alkene (19). This method was successfully tested with alpha-allyl glucosaminide 2 and the dithiol PEG derivatives 3a-c (Scheme 1).

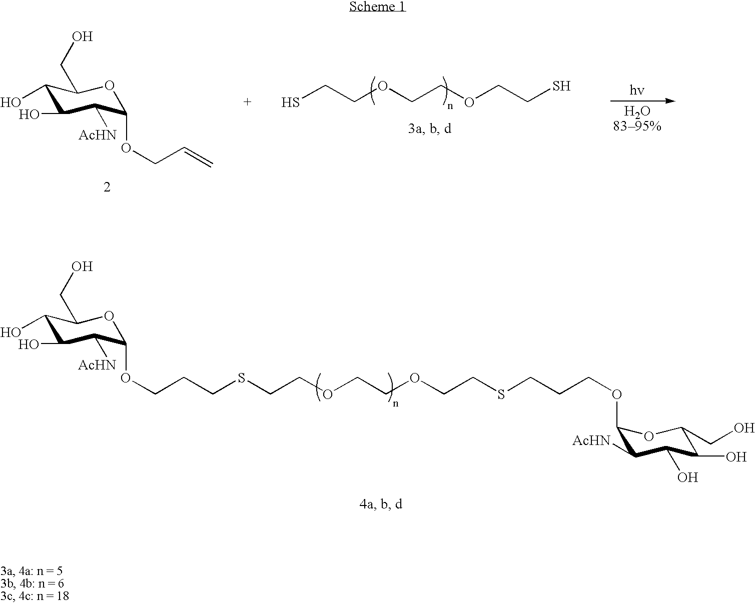

2. Controlling the stereoselectivity of the anomeric alkylation Reaction

The anomeric alkylation method developed by R. R. SCHMIDT (20) and already used in the laboratory (21) was chosen in order to prepare compound 2. Reaction of the alkoxide originating from the hemiacetal 5a with the allyl bromide 6 results exclusively, in dichloromethane, in the beta-stereoisomer.

Surprisingly, it was noted that the addition of tetrabutylammonium iodide reverses the stereochemistry and results mainly in the alpha-isomer in alpha/beta ratios which can range up to 98/2 (22) (Scheme 2). This procedure specific to the invention, resulting unexpectedly in the alpha-isomer, is neither described nor suggested in the prior art.

The reaction conditions for the glucosamine derivative 5a were optimized and applied unmodified to the other substrates. The optimization concerns the temperature, the ratio of the reactants, the concentrations, etc. Thus, these reaction conditions were applied to other sugars and to another electrophile: the benzyl bromide 7. It could thus be shown that this inversion of stereoselectivity of the anomeric alkylation reaction by means of tetrabutylammonium salts was general.

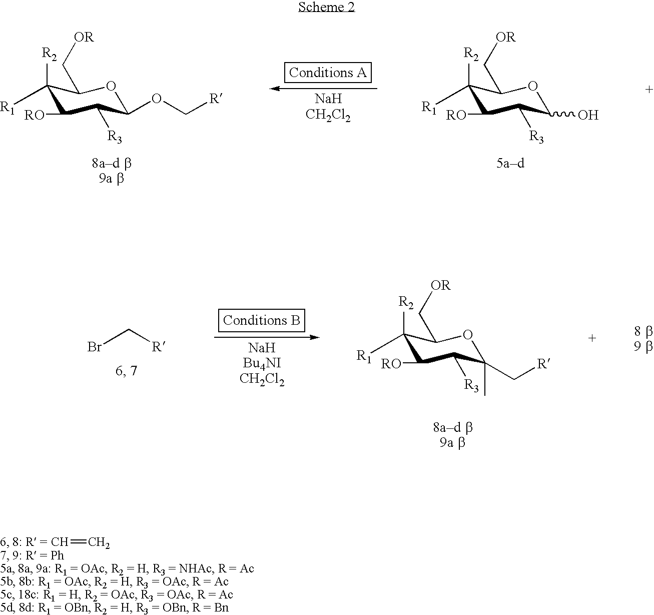

3. Preparation of the heparin fragment Precursor tetra- to octasaccharides

There are many methods for preparing long fragments of heparin or of mimics (23)

Due to the essential step of the process of the invention, it is imperative to prepare fragments having an allyl group in the anomeric position, for the coupling with the spacer, which means that a specific process must be set up. In fact, it was necessary to use a highly convergent method based on the preparation of a suitably protected disaccharide which makes it possible to generate a donor disaccharide and an acceptor disaccharide, so as to prepare a tetrasaccharide, it being possible for the latter to itself serve as a base for the preparation of hexa and octasaccharides. The stereochemical constraints of the oligosaccharide couplings, the desired sulphatation units, and also the need for the allyl group in the anomeric position resulted in disaccharide 10 being envisaged as a basic brick for the synthesis (Scheme 3).

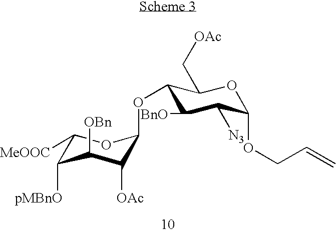

3.1. Synthesis of the Basic disaccharide 10

3.1.2. Novel Access to L-iduronic acid (24)

L-Iduronic acid is a rare sugar that it is impossible to isolate from natural sources in sufficient amounts for synthetic needs. It is therefore essential to have efficient methods for preparing derivatives of this compound (25). Although the addition of organometallics to the aldehyde 12 (26) is reported to occur with a very low diastereoselectivity (27), a study of the addition of carboxylic group precursor nucleophiles to this compound was undertaken in the context of the invention (Scheme 4).

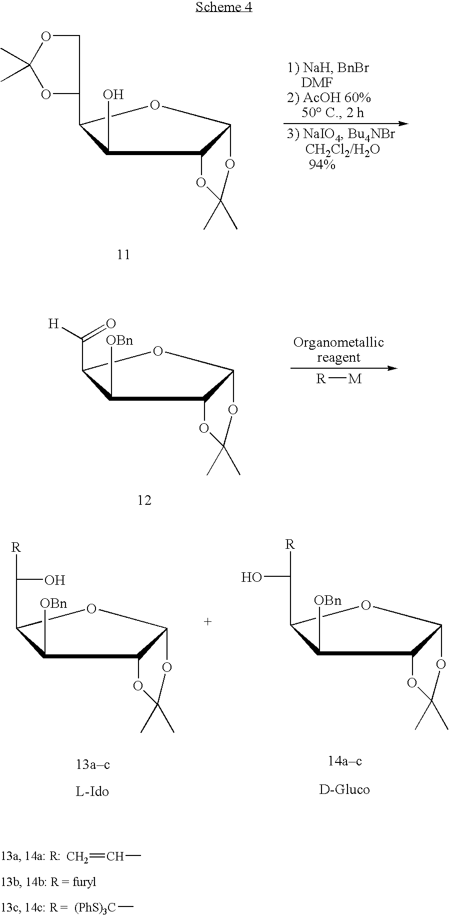

Initially, the addition of vinyl organometallics was studied and it was noted, surprisingly, that, under certain conditions, it was possible to obtain the ido and gluco diastereoisomers 13a and 14b in 50/50 proportions. This result is particularly advantageous from the point of view of the development of combinatorial synthesis of glycosaminoglycan fragments containing glucuronic and iduronic acids.

It was subsequently noted that the proportion of L-ido stereoisomer increased with the hindrance of the nucleophile, so as to reach 100% stereoselectivity with (PhS)₃CLi. This completely stereoselective addition of tris(phenylthio)methyllithium to the aldehyde 12 is the key step in the preparation of the pyranose derivative of L-iduronic acid 17.

Another notable improvement in the process of scheme 5, given in scheme 5a, is the acetylation of the crystals of the compound 15 by carrying out the process at −40° C. in dichloromethane as solvent, with pyridine as base, acetyl chloride as acylating agent and 4-dimethylamino-pyridine as catalyst. Under these conditions, the mixture 17 (α:β, 2/98) is obtained with a 98% yield and the furano derivatives 16 are only present in trace amounts (2%). This improvement is spectacular since, under the conditions previously described (25), the derivatives 16 were obtained with a 40% yield, which required difficult purification and recycling thereof by deacetylation.

The overall yield of the preparation of this synthon, commonly used for preparing heparin fragments, from the diacetone glucose 11, is 65%, which is much higher than the methods previously described, which result in overall yields of 25 to 30% (Scheme 5).

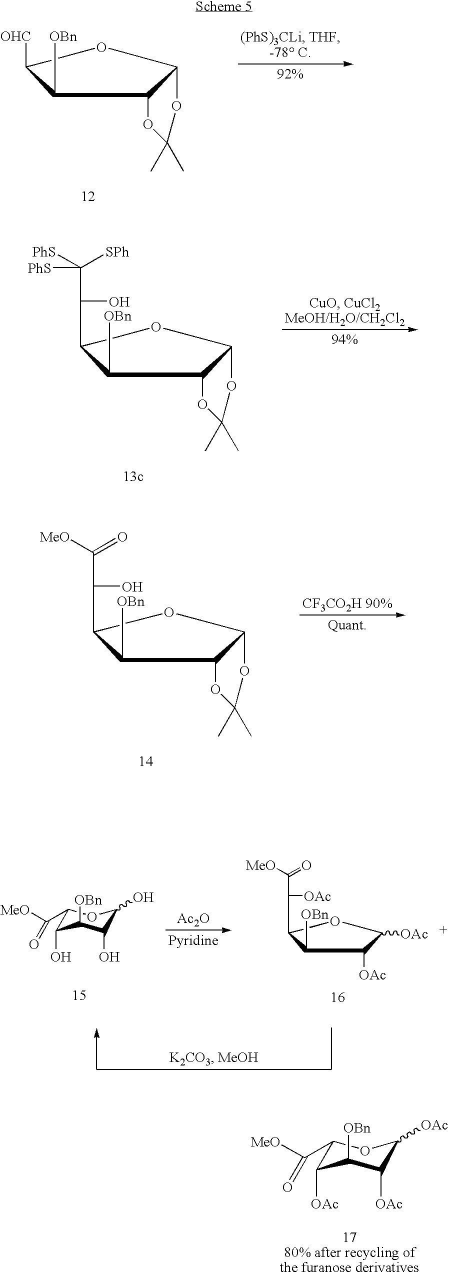

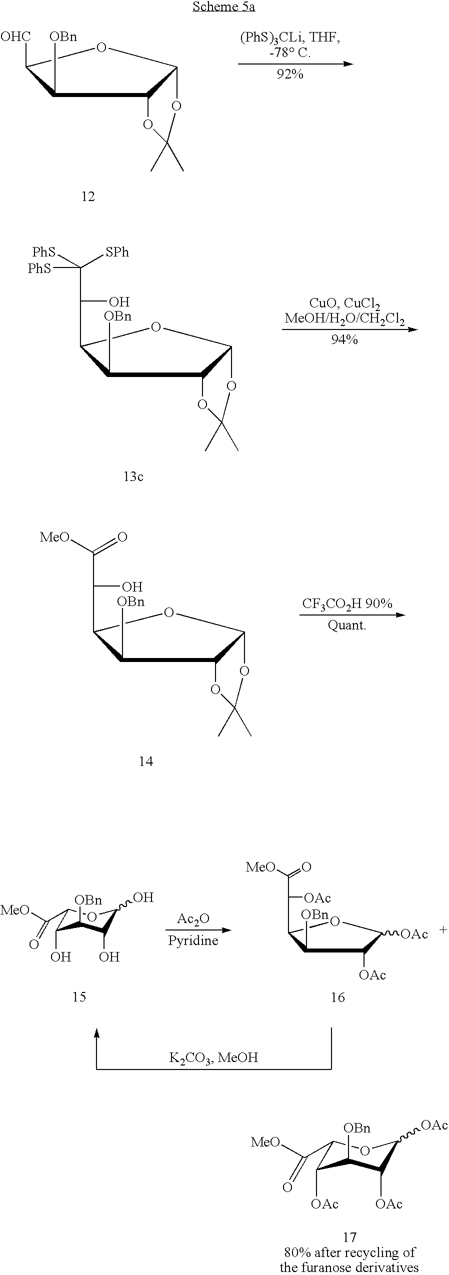

3.1.3. Obtaining the disaccharide 10

Having developed various ways of obtaining L-iduronic acid synthons, it is then necessary to prepare a 2-azidoglucose derivative for preparing the basic block 10. In conventional syntheses involving an azido group in the 2-position, this group is introduced first, followed by the group protecting the anomeric position, via a glycosylation reaction. Since we already have a very effective method of obtaining the alpha allyl-N-acetyl glucosaminide 8a alpha (see Scheme 4), we decided to use the trifyl azide developed by VASELLA (28) for introducing the azido group onto the compound 18, the anomeric position of which is already protected with an allyl group (Scheme 6).

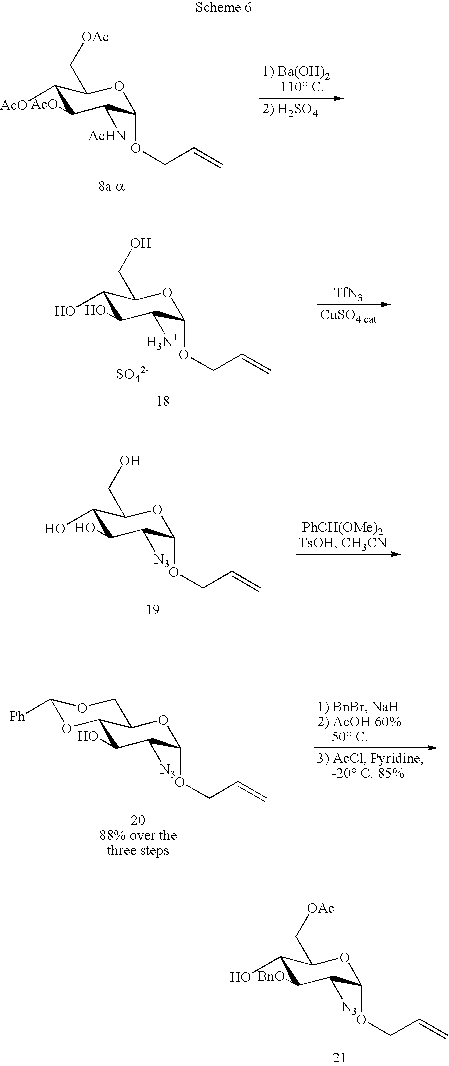

In order to demonstrate that it is possible to use the synthon 13a for preparing heparin-type oligo-saccharides, a specific methodology was developed for converting it to a donor 26, in which all the protective groups of the basic disaccharide 10 are introduced before the coupling reaction (Scheme 7).

This methodology was also applied in part to the glucuronic synthon 14a, and made it possible to prepare a glucuronic acid donor having protective groups similar to 26.

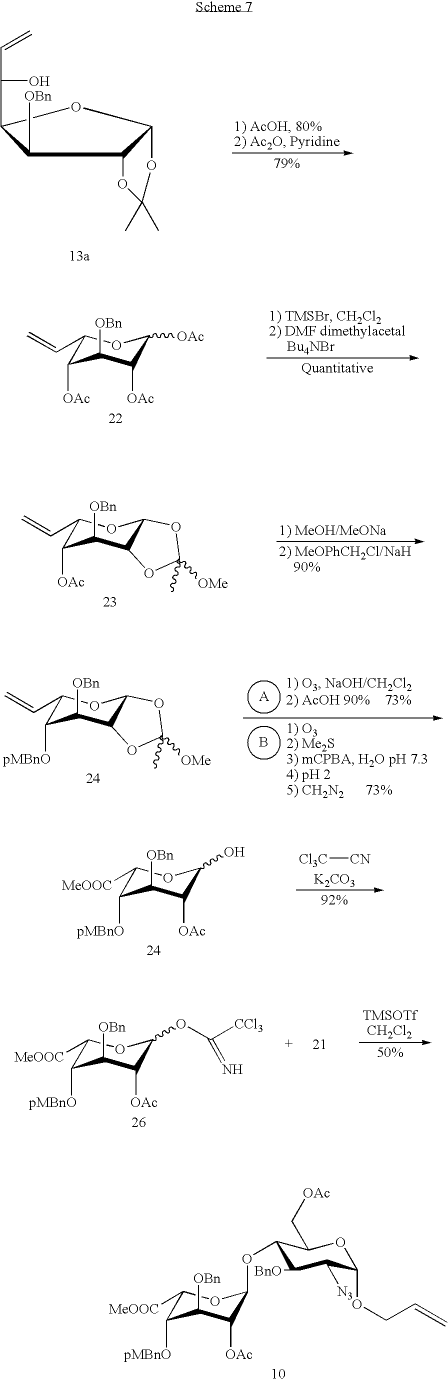

When it is desired to prepare GAGs: glycosaminoglycans possessing only L-iduronic acid, the stereoselective process for obtaining the thioorthoester 13c is much more profitable than the non-stereoselective addition resulting in 13a and 14a. It was shown that the synthon 17 could be converted to donor 27 in a single step.

This compound couples with good yields to the acceptor 21, so as to result in the disaccharide 22, into which the protective groups on the iduronic acid portion still have to be introduced, which process is carried out in a few steps (Scheme 8).

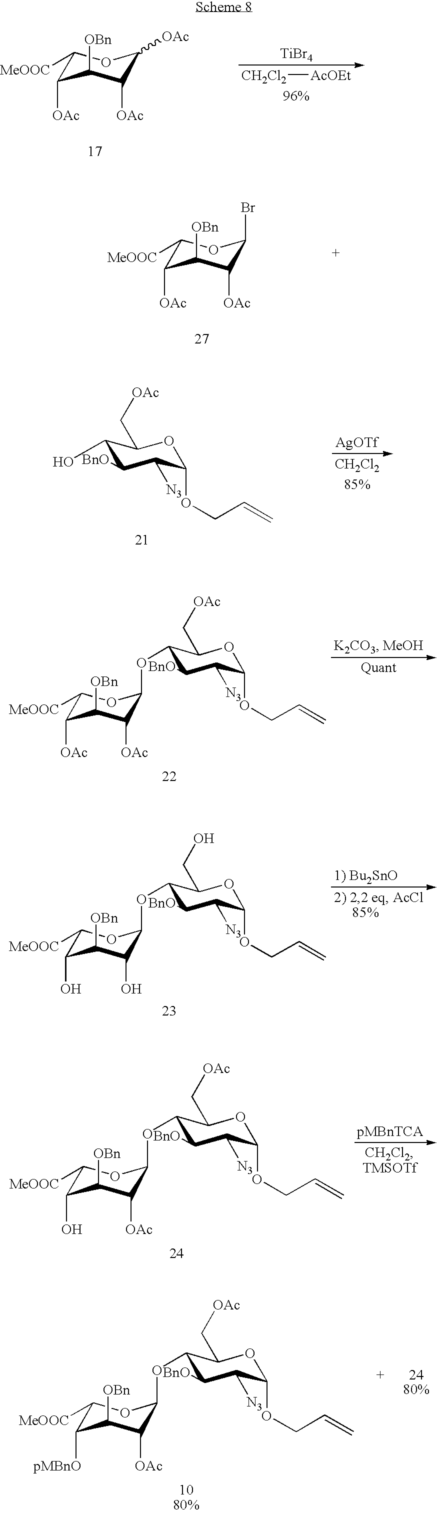

3.2. Effective Strategy for Obtaining the oligosaccharides

Using the disaccharide 10, the acceptor disaccharide 25 is readily prepared by oxidative cleavage of the para-methoxybenzyl group with DDQ: dichlorodicyanoquinone.

The donor disaccharide 26 (29) is, for its part, prepared by isomerization of the allyl to 1-propenyl with an iridium-based catalyst (30), and then hydrolysis of the enol ether thus formed, catalyzed by means of mercury salts, which allows the anomeric position to be freed, which is subsequently activated in the form of trichloroacetimidate (Scheme 9).

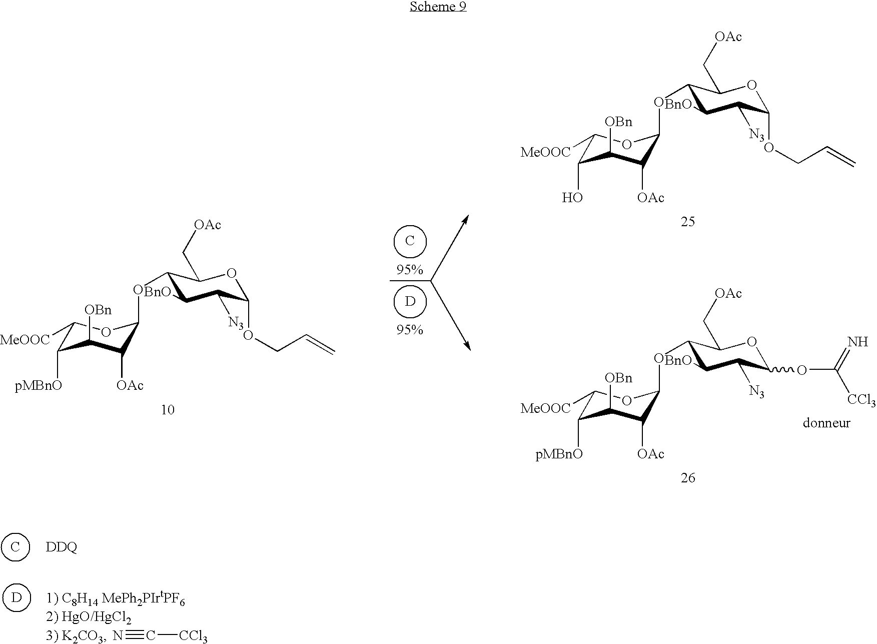

The coupling of the acceptor disaccharide 25 and of the donor disaccharide 26 at −40° C., in the presence of TBDMSOTf, gives, with an excellent yield, the tetrasaccharide 27, with complete alpha-stereoselectivity. By applying to the tetrasaccharide 27 the same operations as to the disaccharide, it is possible to prepare, with very good yields, the acceptor tetrasaccharide 28 and the donor tetrasaccharide 29 (Scheme 10) which make it possible to obtain the hexa- and octasaccharides 30 and 31. This methodology can be extended without any problem to the preparation of a hexadecasaccharide or of a dodecasaccharide.

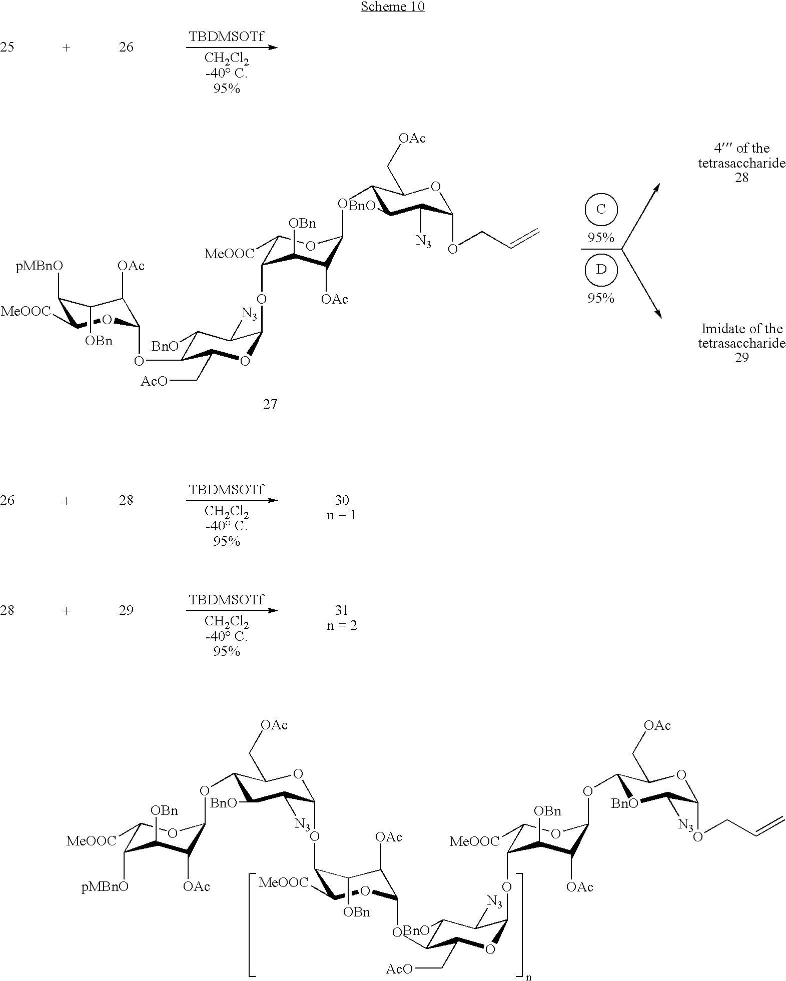

4. Preparation of the neoconjugates

The protected oligosaccharides are converted to partially deprotected and water-soluble compounds by: deacetylation, reduction of the azido function, sulphatation, and then saponification (Scheme 11).

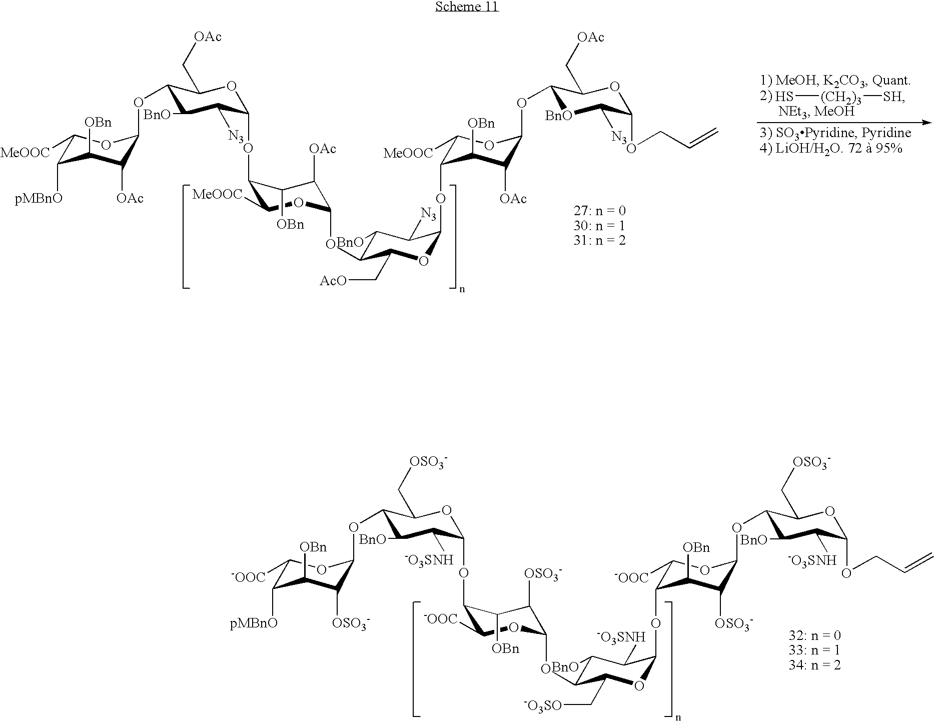

The water-soluble compounds 32-34 ready for coupling to the bisthioPEGs are thus obtained. Under UV irradiation from a medium-pressure lamp, the expected product forms, but accompanied by by-products, probably derived from oxidation of the benzyl groups, which are eliminated by C18 reverse-phase chromatography. The final deprotection of the neoglycoconjugates is carried out by hydrogenolysis, on palladium hydroxide-on-charcoal in the presence of phosphate buffer, pH 7, after oxidation of the thioethers to sulphone in order to prevent catalyst poisoning (Scheme 12).

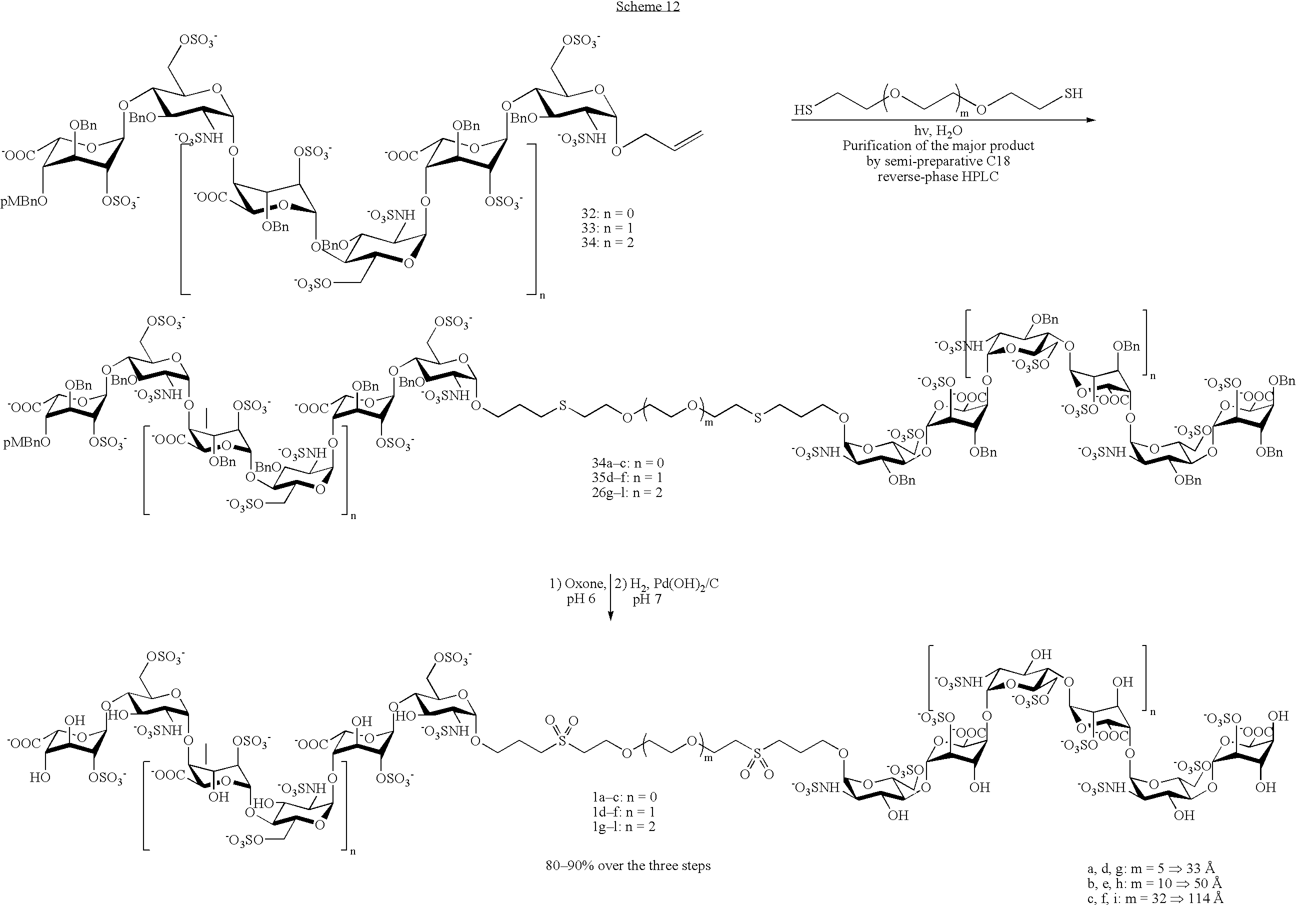

The products thus synthesized were tested for their ability to bind to γ-IFN.

The use of a real-time molecular interaction analysis system (BIAcore) offers the possibility of very rapidly identifying and quantifying the affinity which exists between various molecules.

This device uses an optical detection system, the surface plasmon resonance phenomenon, for measuring the concentration of molecules which have reacted at the surface of a biosensor (“sensor chip”). Biotinylated heparin is immobilized on a “sensor chip” pre-activated with streptavidin. It is then possible to measure the interaction of γ-IFN, injected at a continuous flow at the surface of this sensor, with the immobilized heparin. The interaction between the heparin and the γ-IFN results in a change in mass at the surface of the sensor, which is recorded as a function of time. In a second step, the γ-IFN is pre-incubated with the various products synthesized, and the complexes are then injected at the surface of the biosensor. The ability of the products tested to inhibit the γ-IFN/heparin interaction is then measured.

The results are disclosed in FIG. 2, and indicate that a molecule containing a spacer arm of 5 nm (50 Å), connecting two octasaccharides, has a very high affinity for the cytokine.

The same is true for the molecules containing a spacer arm of 33 Å and of 114 Å, which also have the ability to interact with γ-IFN, but with affinities that are, however, lower.

The invention will now be described with reference to the following example, given by way non-limiting illustration.

### EXAMPLE 1

This example describes the synthesis of compounds according to the invention: i.e. of interferon-binding neoglycoconjugates, in which the spacer group or arm is derived from poly(ethylene glycol)s of variable lengths and the two end oligosaccharides consist of trisulphated disaccharides. The synthesis starting product is the compound 8α, prepared according to A. LUBINEAU, S. ESCHER, J. ALAIS, D. BONNAFFE, Tetrahedron Lett. 1997, 38, 4 087-4 090, i.e. procedure 1 below:

Procedure 1

Commercially available glucosamine hydrochloride is peracetylated in a mixture of acetic anhydride (20 equivalents) and of pyridine (30 equivalents). After evaporation of the reactants and coevaporation with toluene, the mixture is crystallized from an ethyl acetate/petroleum ether mixture so as to give peracetylated glucosamine with a 90% yield. The recovered crystals are treated with 1.1 equivalents of hydrazine acetate in anhydrous THF (0.3 M concentration) for 20 h at 20° C. The THF is then evaporated at ambient temperature under reduced pressure, the residue is coevaporated with toluene and the residual oil is directly chromatographed on silica gel with an EtOAc/petroleum ether/CH₂Cl₂ (8/1/1 to 8/0/2) mixture. The composition that is free in the anomeric position is obtained with a 75% yield. It is then solubilized in the required volume of CH₂Cl₂, so as to have a concentration of 0.5 M, and added dropwise to a mixture of tetrabutylammonium bromide (2 eq.), of NaH (1.5 eq.) and of allyl bromide (20 eq.) cooled to −20° C. The temperature is then allowed to return to 20° C. over 12 h and the reaction is stopped by adding 0.5 equivalent of acetic acid. The reaction medium is evaporated under reduced pressure and the residue is directly chromatographed on silica (eluent: 1/0 to 7/3 toluene/acetone) so as to give the compound 8a α with an 88% yield.

The compound 8a α is treated with eight equivalents of Ba(OH)₂ in water at 100° C. overnight, the pH is then lowered to 3 by adding sulphuric acid, the mixture is centrifuged so as to precipitate the barium sulphate, and the supernatant is collected, then evaporated under reduced pressure and coevaporated with water so as to remove the acetic acid formed. The salt 18 is neutralized with potassium carbonate and directly treated with TfN₃, according to the protocol described in P. B. ALPER, S. C. HUNG, C. H. WONG, Tetrahedron Lett. 1996, 37, 6 029-6032, i.e. according to procedure 2 below:

Procedure 2

15 equivalents of NaN₃ are dissolved in the minimum amount of water required, and the solution is cooled to 0° C. and then an equivalent volume of dichloromethane and 2 equivalents of triflic anhydride are added dropwise. After vigorous stirring for 2 h at 0° C., the mixture is allowed to separate by settling out and the organic phase is then recovered and washed with an equivalent volume of saturated sodium bicarbonate solution. The organic phase is then directly added to an aqueous solution of the salt 18 and of 0.01 equivalent of copper sulphate. The minimum amount of methanol required to obtain a homogeneous mixture is then added. After reaction for 4 h at 20° C., one equivalent of butylamine is added in order to destroy the excess triflyl azide.

The resulting solution is deposited onto 70-200μ silica gel by evaporation and the compound 19 is eluted with an 8/2 MeOH/CH₂Cl₂ mixture.

After evaporation under reduced pressure, the residue is solubilized in anhydrous acetonitrile and treated with benzaldehyde dimethyl acetal in the presence of camphorsulphonic acid. After two hours at ambient temperature, the solution is neutralized by adding an aqueous sodium bicarbonate solution, and the compound 20 is extracted with diethyl ether. After purification by chromatography on silica gel (petroleum ether/EtOAc), the compound 20 is treated with benzyl bromide in the presence of NaH in DMF. After dilution with diethyl ether, washing of the organic phase with water and evaporation, the residue is treated at 50° C. with a 60% acetic acid solution for two hours. After evaporation and coevaporation with toluene, the residue is chromatographed on silica gel (petroleum ether/EtOAc). The diol thus obtained is solubilized in anhydrous pyridine and one equivalent of acetyl chloride is added at −20° C., and the temperature is then allowed to return to 0° C. The reaction is complete after an overnight period. The pyridine is then evaporated off under reduced pressure and the residue is solubilized in diethyl ether, and then the organic phase obtained is washed with a dilute HCl solution, water and a sodium bicarbonate solution. The organic phase is then dried, evaporated, and chromatographed on silica gel with a mixture of petroleum ether and of EtOAc. The compound 21 is thus obtained with an overall yield of 75% from 8a α.

Moreover, the derivative 17 is prepared according to Lubineau A.; Gavard O.; Alais J.; Bonnaffe E., Tetrahedron Lett. 2000, 307-311, i.e. according to procedure 3 below:

Procedure 3:

Commercially available diacetoneglucose, solubilized in anhydrous dimethylformamide (0.5 M concentration), is treated with 1.2 equivalents of benzyl bromide and 1.3 equivalents of NaH. After 1 h at 20° C., the excess NaH is destroyed with isopropanol. The reaction mixture is then diluted with ethyl ether and the organic phase is washed three times with water. After evaporation, the residue is taken up with 60% acetic acid so as to achieve a concentration of 0.1 M. After two hours at 50° C., the mixture is evaporated and then coevaporated with toluene under reduced pressure. Chromatography on silica gel (eluent: 9/1 to 2/8 petroleum ether/ethyl acetate) makes it possible to obtain the desired diol with a 98% yield. This diol is solubilized in dichloromethane (0.25 M concentration), and then the same volume of water, 0.5 equivalent of tetrabutyl-ammonium hydrogen sulphate, and 0.5 equivalent of NaHCO₃ are added, followed, after having lowered the temperature to 0° C., by 2 equivalents of NaIO₄, in small portions. The temperature is then allowed to return to ambient temperature and the mixture is then left stirring for 1 h. After separation by settling out, the organic phase is recovered and is evaporated and then taken up with ethyl ether. The organic phase is washed three times with water, dried over magnesium sulphate, filtered and evaporated. The residue is filtered on silica gel (eluent: ethyl acetate) and the compound 12, obtained quantitatively, is directly used, after evaporation and coevaporation with toluene, in the subsequent step. A solution of trisphenylthiomethyl-lithium is prepared in the following way: 1.2 equivalents (relative to the aldehyde) of trisphenylthiomethane are solubilized in the amount of THF required to obtain a concentration of 0.8 M, the temperature is lowered to −78° C. and the mixture is stirred mechanically, and then 1.1 equivalents (relative to the aldehyde) of n-butyllithium in solution in hexane are added. A yellow precipitate appears and the stirring is maintained at −78° C. for 1 h 30 min. The aldehyde, solubilized in THF (0.6 M concentration) is then added dropwise. The mixture is stirred at −78° C. for one hour and then the temperature is allowed to return to ambient temperature over 16 h and the reaction is stopped by adding a saturated ammonium chloride solution. The resulting solution is extracted three times with ethyl ether, and the organic phase is dried over magnesium sulphate, evaporated, and then chromatographed on silica gel (eluent: 9/1 to 6/4 petroleum ether/ethyl acetate). The compound 13c is thus obtained with a 92% yield.

The trisphenylthioorthoester 13c obtained above is solubilized in the volume of methanol required to obtain a concentration of 0.05 M; a 1/10th volume of water and a 1/10th volume of dichloromethane are then added, followed by 1.7 equivalents of CuO and 4 equivalents of CuCl₂. After one hour at ambient temperature, the mixture is filtered through Celite 545 and evaporated at ambient temperature under reduced pressure. The residue is taken up in CH₂Cl₂ and the organic phase is washed with a saturated NaCl solution, filtered through phase-separating paper and evaporated. The mixture is purified by chromatography on silica (eluent: 8/2 to 5/5 petroleum ether/EtOAc). The product 14 is thus obtained with a 94% yield. The product 14 is then treated with a 90% trifluoroacetic acid solution for 30 minutes at ambient temperature (initial concentration of the reactant: 0.35 M). The reaction medium is evaporated under reduced pressure and then coevaporated twice with water. An oil. is thus obtained, which crystallizes. These crystals of the compound 15 are suspended in dichloromethane (0.2 M concentration) and the temperature is lowered to −40° C. 9 equivalents of pyridine, 0.01 equivalent of 4-dimethylaminopyridine and 5 equivalents of acetyl chloride are then added. After stirring for 1 h at −40° C., the temperature is allowed to return to ambient temperature, the mixture is diluted with dichloromethane and the organic phase is washed with a saturated solution of NaHCO₃, water, 1 M sulphuric acid and water. The mixture 17 (α/β 2/98) is thus obtained with a 98% yield.

The derivative 17 is converted to the donor 27 according to Jacquinet J. C.; Petitou M.; Duchaussoy P.; Lederman I.; Choay J.; Torri G.; Sinay P., Carbohydrate Res. 1984, 130, 221, i.e. according to procedure 4 below:

Procedure 4:

The mixture 17 is dissolved in anhydrous dichloromethane (0.1 M concentration) and a 1/10 th volume of anhydrous ethyl acetate is then added and the mixture is allowed to react for 24 h in the presence of 1.3 equivalents of TiBr₄. The mixture is diluted with CH₂Cl₂ and washed with ice-cold water. The organic phase is filtered through silicone paper and then concentrated. The product 27 thus obtained is used directly in the subsequent step.

The coupling of 21 (1.2 equivalents) with 27 is carried out in dichloromethane at 20° C., in the presence of 4 Å sieve and of silver triflate (1.2 equivalents) and results, after chromatography on silica gel with the petroleum ether/EtOAc mixture, in the disaccharide 22, with an 85% yield.

The compound 22 is deacetylated in anhydrous methanol in the presence of K₂CO₃, and after neutralization with DOWEX® 50×8 200 H⁺ resin, filtration and evaporation, the residue is suspended in benzene and then 2.2 equivalents of Bu₂SnO are added. After 2 hours of azeotropic entrainment of water, 3 equivalents of triethylamine and 2.2 equivalents of freshly distilled acetyl chloride are added. A mixture of 3 products is then obtained, the major one of which is the compound 24 accompanied by the compounds acetylated in the 6/4′ and 6/2′/4′ position.

After chromatography on silica gel with the petroleum ether/EtOAc mixture, the by-products are deacetylated and again subjected to the acylation reaction: the compound 24 is thus obtained after recycling, with an 85% yield.

Treatment of this compound 24 with 2 equivalents of para-methoxybenzyl alcohol trichloroacetimidate in dichloromethane in the presence of 0.1 equivalent of trimethylsilyl triflate makes it possible to obtain the compound 10 with an 80% yield, accompanied by 20% of 24 which has not reacted. These two products 10 and 24 can be readily separated by chromatography on silica gel with the petroleum ether/EtOAc mixture.

The basic disaccharide 10 is converted with a 95% yield into the donor disaccharide 26 (synthesized via another route in Tabeur C.; Mallet J. M.; Bono F.; Herbert J. M.; Petitou M.; Sinay P., Bioorg., Med. Chem. 1999, 7, 2 003-2 012 by isomerization of the allyl, in the presence of C₈H₁₄MePh₂PIr¹PF⁶, and cleavage of the enol with mercury salts, as is described by Oltvoort J. J.; Van Boeckel C. A. A.; Koning J. H.; Van Boom J. H., Synthesis 1981, 305-308, i.e. according to procedure 5 below:

Procedure 5:

The disaccharide 10 is solubilized in anhydrous THF so as to have a concentration of 0.06 M. The solution is degassed under vacuum, and then 0.013 equivalent of C₈H₁₄MePh₂PIr^{I}PF⁶ is added. The mixture is again degassed and left in contact with dihydrogen for 2 minutes and, finally, degassed again and then placed in an argon atmosphere. After stirring for two hours at ambient temperature, the mixture is evaporated and then taken up in the volume of acetone required to obtain a concentration of 0.05 M; 1.2 equivalents of HgO and 1.1 of HgCl₂ are then added. After stirring for two hours at ambient temperature, the mixture is filtered through Celite 545, evaporated, and taken up with ethyl ether. The ethereal phase is washed with a 10% KI solution and then with water, dried over magnesium sulphate, filtered, evaporated and, finally, chromatographed on silica gel (eluent: 8/2 to 6/4 toluene/EtOAc), to give the disaccharide free in the anomeric position, with a 97% yield.

Next, a treatment is carried out in dichloromethane with 6 equivalents of trichloroacetonitrile and 2 equivalents of K₂CO₃, followed by chromatography on silica gel with the mixture of petroleum ether/EtOAc 1% NEt₃. The disaccharide 10, treated with 1.5 equivalents of DDQ in dichloromethane saturated with water, gives, after washing with the organic phase, with a saturated NaHCO₃ solution and chromatography on silica gel with the petroleum ether/EtOAc mixture, the acceptor disaccharide 25 with a 95% yield.

The coupling of the acceptor disaccharide 25 and of the donor disaccharide 26 (1.3 equivalents) is carried out in dichloromethane at −40° C. in the presence of 4 Å sieve and of tert-butyldimethylsilyl triflate (0.2 equivalent). After addition of the catalyst, the reaction is left for 30 minutes at −40° C., and then the temperature is allowed to return to 0° C., at which temperature the reaction is stopped by adding 0.2 equivalent of triethylamine. Direct chromatography on silica gel of the reaction mixture, with the CH₂Cl₂/EtOAc/petroleum ether mixture, makes it possible to isolate the tetrasaccharide 27 with a 95% yield.

This tetrasaccharide 27 is then converted into the acceptor tetrasaccharide 28 (90% yield) and the donor tetrasaccharide 29 (95% yield) using the same reaction sequences as for the disaccharide 10. Using the same conditions as for the preparation of the tetrasaccharide 27, the couplings of the donor disaccharide 26 with the acceptor tetrasaccharide 28 and of the donor tetrasaccharide 29 with the acceptor tetrasaccharide 28 gives the hexasaccharide 30 and the octasaccharide 31, with 95% yields.

The oligosaccharides 27, 30 and 31 are deacetylated in anhydrous methanol in the presence of 0.5 equivalent of anhydrous K₂CO₃. After neutralization of the reaction media with DOWEX 50×8 200 H⁺ and evaporation, the products are purified by chromatography on silica gel and obtained with 86 to 95% yields. The reduction of the azido groups is carried out with propanedithiol (2 equivalents per azido group) in methanol in the presence of triethylamine (2 equivalents per azido group). After evaporation of the methanol by argon sweeping, the residues are chromatographed on silica gel (CH₂Cl₂/MeOH) to give the aminated compounds with 90% to 80% yields.

These compounds are then sulphated with the pyridine. SO₃ complex (5 equivalents per function to be sulphated) in pyridine for 16 hours at 20° C., followed by 24 hours at 55° C. The excess sulphating agent is destroyed with 16 equivalents of methanol and the mixture is directly chromatographed on Sephadexo LH-20 (1/1 CH₂Cl₂/MeOH), and then on a C18 reverse-phase column (MeOH/5 mM AcOH-Net₃ buffer, pH 7.0). After exchange on Biorad® AG 50 W×8 200 Na⁺ resin, the sulphated oligosaccharides are obtained with yields ranging from 80 to 95%. Saponification is carried out for 48 hours at 37° C., in the presence of aqueous lithium hydroxide (26 equivalents per ester to be saponified) and of aqueous hydrogen peroxide solution (3 equivalents relative to the lithium hydroxide). The pH is then brought to 5 by adding acetic acid (0.75 equivalent relative to the lithium hydroxide) and the mixture is purified by chromatography on a C18 reverse-phase column (MeOH/10 mM AcOH—NEt₃ buffer, pH 7.0). After exchange on Biorad® AG 50 W×8 200 Na⁺ resin, the oligosaccharides 32 to 34, in which, respectively, n=0, 1 and 2, are obtained with 90% to quantitative yields.

The couplings of the oligosaccharides 32 to 34 (2.5 equivalents) to the polyethylene glycol-derived dithiol spacers of variable lengths: namely, m=5, 10 and 32, at a concentration of 0.2 M in water, are carried out under ultraviolet irradiation at 365 nM or by heating in the presence of a radical initiator.

The mixture is directly treated with 8 equivalents of oxone, i.e. with a solution at 0.2 M, brought to pH 6 by adding K₂HPO₄. After reaction for 4 hours at 20° C., the excess oxidizing agent is reduced by adding a sodium thiosulphate solution and the mixture is directly purified by semi-preparative C18 reverse-phase HPLC (CH₃CN/5 mM AcOH—NEt₃ buffer, pH 7.0). After exchange on Biorad® AG 50 W×8 200 Na⁺ resin, the oligosaccharides are resolubilized in 100 μl of 40 mM sodium phosphate buffer, pH 7, and placed under an atmospheric hydrogen pressure in the presence of one mass-equivalent of 20% palladium hydroxide-on-charcoal. After reaction for 48 hours at 20° C., the samples are filtered through Celite 545, and then desalified on Biogel® 10 DG (H₂O). The compounds IIIa-i are thus obtained with an 80 to 90% yield over these three steps. In parallel, the oligosaccharides 32 to 34 were debenzylated quantitatively under the same conditions. For the compounds 1a to 1c, n=0; for the compounds 1d to 1f, n=1; and for the compounds 1g to 1i, n=2. For the compounds 1a, 1d and 1g: m=5 and the length of the spacer arm is 33 Å; for the compounds 1b, 1c and 1h: m=10 and the length of the spacer arm is 50 Å; and for the compounds 1c, 1f and 1i: m=32 and the length of the spacer arm is 114Å.

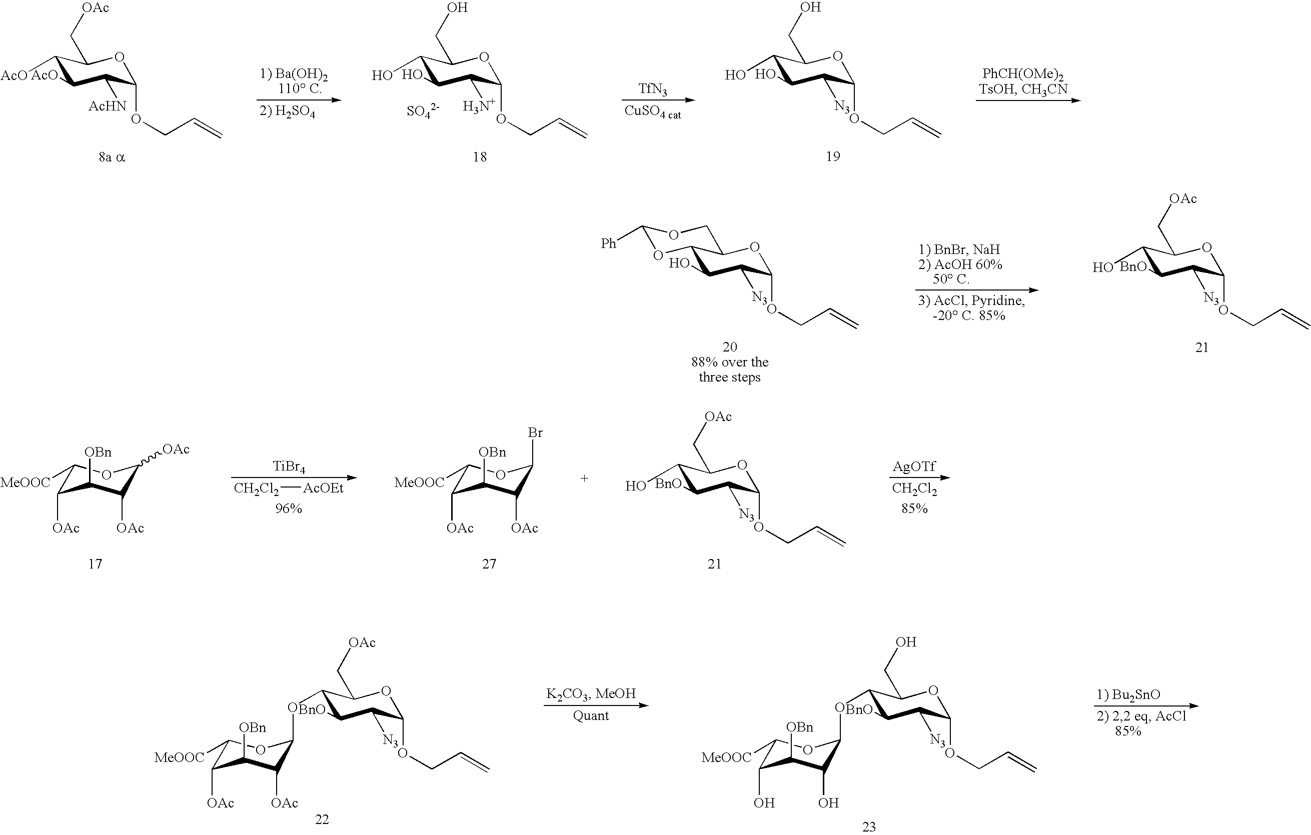

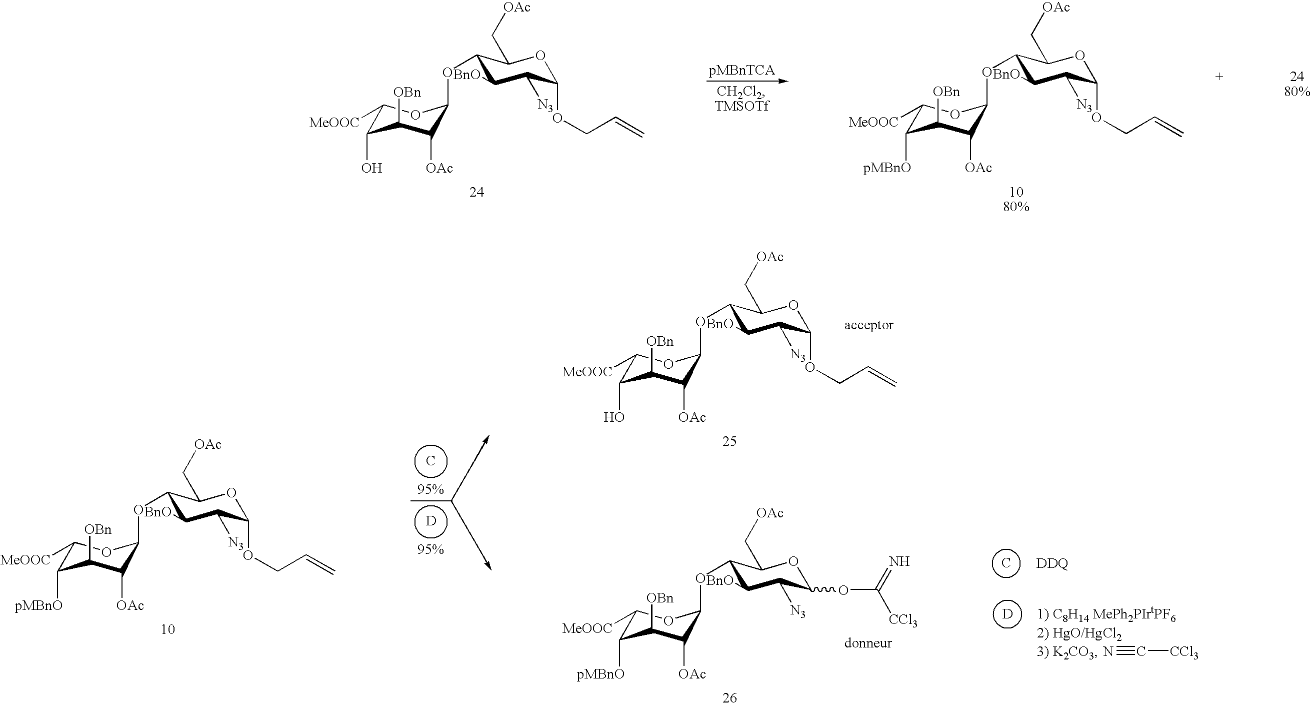

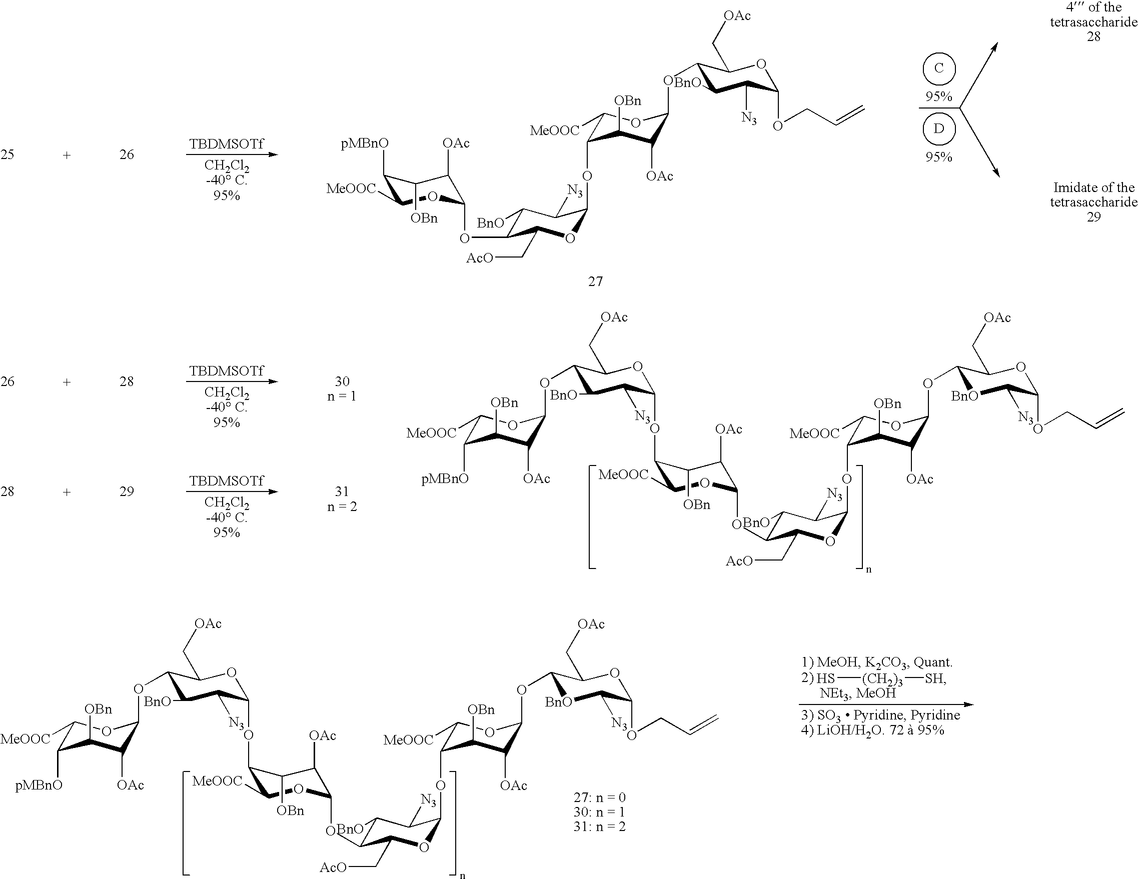

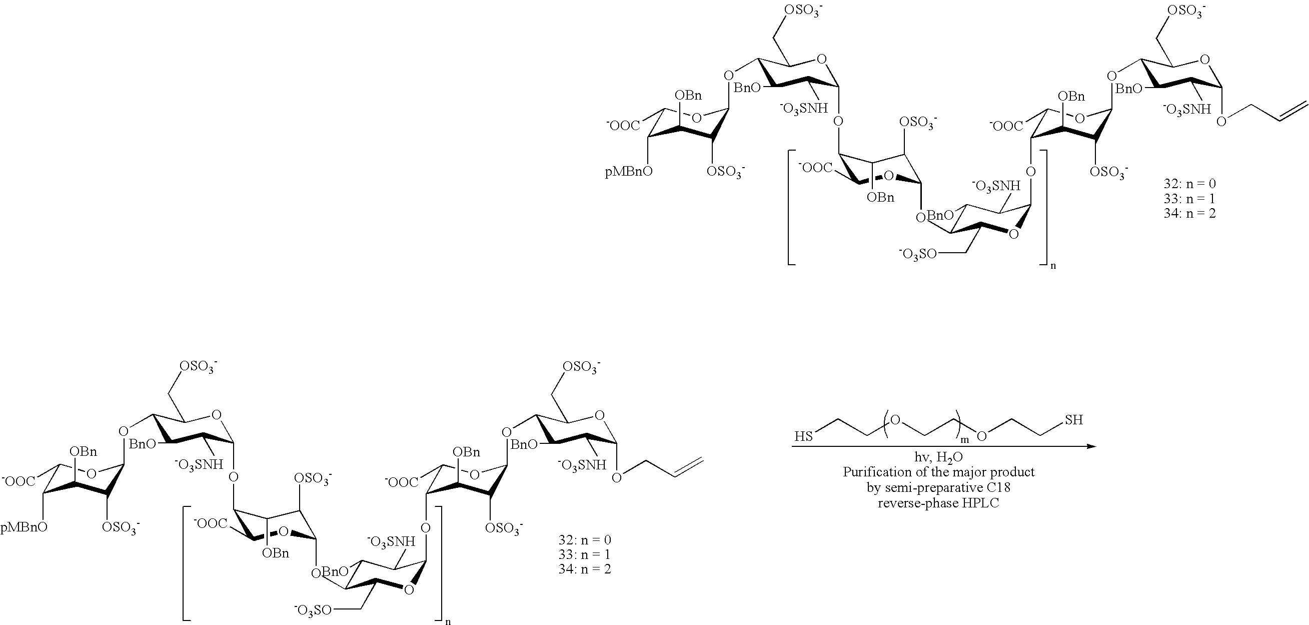

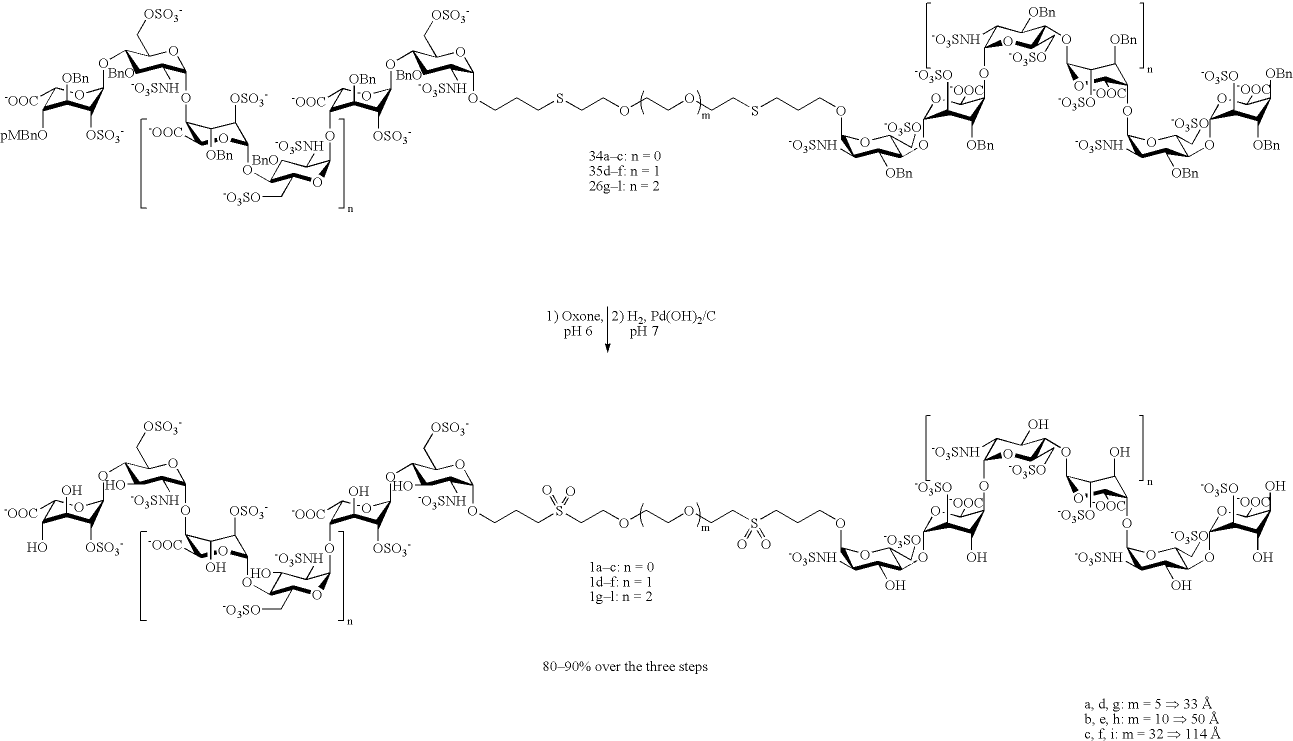

### REFERENCES

1. Billiau A., Adv. Immunol. 1996, 65: 61-130.
2. Boutin C., E. Nussbaun, I. Monnet, J. Bignon, R. Vanderschueren, J. C. Guerin, O. Menard, P. Mignot, G. Dabouis and J. Y. Douillard, 1994, Cancer 74 : 2 460-2 467, Escudier B., F. Farace, E. Angevin, F. Triebel, S. Antoun, B. Leclercq, M. Brandely, A. Aboudaram, G. Nitenberg and T. Hercend., 1993, Eur. J. Cancer 29A: 724-728. Jett, J. R., A. W. Maksymiuk, J. Q. Su, J. A. Mailliard, J. E. Krook, L. K. Tschetter, C. G. Kardinal, D. I., Twito, R. Levitt, and J. B. Gerstner. 1994. J. Clin. Oncol. 12: 2321-2 326.
3. Gallin J. I., 1991, New. Engl. J. Med. 324: 509-516.
4. Cannon, G. W., R. D. Emkey, A. Denes, S. A. Cohen, P. A. Saway, F. Wolfe, A. M. Jaffer, A. L. Weaver, B. J. Manaster, and K. A. McCarthy, 1993. J. Rheumatol. 20: 1867-1873.
5. Czarniecki, C. W., and G. Sonnenfeld., 1993. APMIS 101: 1-17. Murray, H. W., 1994. Am. J. Med. 97: 459-467.
6. Kakumu, S, T. Ishikawa, M. Mizokami, E. Orido, K. Yoshioka, T. Wakita, M. Yamamoto., 1991., J. Med. Virol. 35: 32-37.
7. Freundlich B, Jimenez S A, Steen V D, Medsger T A, Szkolnicki M, Jaffe H S., 1992., Arthritis Rheum. 35: 1134-1142.
8. Todd P. A. and Goa K. L., 1992., Grugs 43: 111-122.
9. Froyen G. and Billiau A., 1997, Biotherapy 10: 49-57.
10. Ozmen L., Roman D., Fountoulakis M., Schmid G., Ryffel B., Garotta G. J., Interferon Res, 1994, 14: 283-4. Ozmen L., Roman D., Fountoulakis M., Schmid G., Ryffel B., Garotta G., Eur. J. Immunol., 1995, 25: 6-12.
11. Lortat-Jacob H., H. K. Kleinman, and J. A. Grimaud, 1991, J. Clin. Invest., 87: 878-883. Lortat-Jacob H. and J. A. Grimaud, 1991, Cell. Mol. Biol. 37: 253-360. Lortat-Jacob H. and J. A. Grimaud, 1991, FEBS Lett. 280: 152-154.
12. Lortat-Jacob H. Baltzer F. and Grimaud J. A., Heparin decreases the blood clearance of interferon-gamma and increases its activity by limiting the processing of its C-terminal sequence, 1996, J. Biol. Chem., 271, 16 139-16 143.
13. Lortat-Jacob H., Brisson C., Guerret S. and Morel G., 1996, Cytokine 8, 557-566.
14. Dianzani F., 1992, J. Interferon Res., 5: 109-118.
15. Halme M., Maasilta P., Repo H., Ristola M., Taskinen E., Mattson K., Cantell K., 1995, Int. J. Radiat. Oncol. Biol. Phys., 31: 93-101.
16. Lack, G., Bradley K. L., Hamelmann E., Renz H., Loader J. Leung D. Y., Larsen G., Gelfand E. W., 1996, J. Immunol. 157: 1 432-9.
17. Short S. M., Paasch B. D., Turner J. H., Weiner N., Daugherty A. L., Mrsny R. J., 1996, Pharm/Res. 13: 1 020-7.
18. Lortat-Jacob H., J. Turnbull and J. A. Grimaud, 1995, Biochem., J 310: 497-505.
19. Lee, R. T., Lee Y. C., Carbohydrate Res., 1974, 37, 193-201.
20. (a) Schmidt R. R., Angew. Chem. Int. Ed. Engl., 1986, 25, 212-235, (b) Klotz W., Schmidt R. R., Liebigs Ann. Chem., 1993, 683-690 (c) Klotz W., Schmidt R. R. J., Carbohydr. Chem., 1994, 13, 1 093-1 101, (d) Terjung A., Jung K. H., Schmidt R. R., Carbohydr. Res., 1997, 297, 229-242.
21. Lubineau A., Bienayme H., Le Gallic J. J., Chem. Soc. Chem. Commun., 1989, 1 918-1 919.
22. Lubineau A., Escher S., Alais J., Bonnaffe D., Tetrahedron Lett., 1997, 38 : 4 087-4 090.
23. Sakairi N., Basten J. E. M., Gijsbert A., van der Marel G. A., van Boeckel C. A. A., Van Boom, J. H., Chem. Eur. J., 1996, 2, 1 007-1 013. Petitou M., Duchaussoy P., Bernat A., Hoffmann P., Herbert J. M., Bioorg. Med. Chem. Lett., 1997, 7, 2 067-2 070, Petitou M., Duchaussoy P., Driguez P. A., Jaurand G., Herault J. P., Lormeau J. C., van Boeckel C. A. A., Herbert, J. M. Angew. Chem. Int. Ed. Engl., 1998, 37, 3 009-3 014, LEI P.-S., Duchaussoy P., Sizun P., Mallet J. M., Petitou M., Sinay P., Bioorg. Med. Chem., 1998, 6, 1 337-1 346, Basten J. E. M., Dreef-Tromp, C. M., de Vijs B., van Boeckel C. A. A. Bioorg. Med. Chem. Lett., 1998, 8, 1 201-1 206, Duchaussoy P., Jaurand G., Driguez P. A., Lederman I., Gourvenec F., Strassel J. M., Sizun P., Petitou M., Herbert J. M., Carbo-hydrate Res., 1999, 317, 63-84, Duchaussoy P., Jaurand G., Driguez P. A., Lederman I., Ceccato M. L., Gourvenec F., Strassel J. M., Sizun P., Petitou M., Herbert J. M., Carbohydrate Res., 1999, 317, 84-99, Koshida S., Suda Y., Sobel M., Ormsby J., Kusumoto, Bior. Med. Chem. Lett., 1999, 9, 3 127-3 132, Kovensky J. Duchaussoy P., Bono F., Salmivirta M., Sizun P., Herbert J. M., Petitou M., Sinay P., Bioorg. Med. Chem. Lett., 1999, 9, 1567-1580, La Ferla B., Lay L., Guerrini M., Poletti M., Panza L., Russo, Tetrahedron 1999, 55, 9867-9880.
24. Lubineau A., Gavard O., Alais J., Bonnaffe D., Tetrahedron Lett., 2000, 307-311.
25. (a) Macher I., Dax K., Wanek E., Weidmann H., Carbohydrate Res., 1980, 80, 45-51, (b) Jacquinet J. C., Petitou M., Duchossoy P., Lederman I., Choay J., Torri G., Sinay P., Carbohydrate Res., 1984, 130, 183-193, (c) Baggett N., Samra A. K., Carbohydrate Res., 1984, 127, 149-153, (d) Medakovic D., Carbohydrate Res., 1994, 253, 299-300, (e) Dromowicz M., Koll P., Carbohydrate Res., 1998, 308, 169-171, (f) Hinou H., Kurosawa H., Matsuoka K., Terunuma D., Kuzuhara H., Tetrahedron Lett., 1999, 40, 1511-1504. And references quoted (g) Adinolfi M., Barone G., de Lorenzo G., Iadonisi A., Synlett, 1999, 336-338, (h) Ojeba R., de Paz, j. L., Martin-Lomas M., Lassaletta J. M., Synlett, 1999, 1316-1318. Takahashi H., Hitomi Y., Iwai Y., Ikegami, S. J. Am. Chem. Soc., 2000, 122, 2 995-3 000.
26. Horton D., Swanson F.O., Carbohydrate Res., 1970, 14, 159-171, Horton D., Tsai J. H., Carbohydrate Res., 1977, 58, 89-108.
27. Danishefsky S. J., Deninno M. P., Philips G. B., Zelle R. E., Lartey P. A., Tetrahedron, 1986, 42, 2 809-2 819.
28. (a) Vasella A., Witzig C. Chiara J. L., Martin-Lomas M., Helvetica Chim. Acta 1991, 74, 2 073-2 077, (b) Alper P. B., Hung S. C., Wong C. H., Tetrahedron Lett. 1996, 37, 6 029-6 032.
29. Tabeur C., Mallet J. M., Bono F., Herbert J. M., Petitou M., Sinay P., Bioorg. Med. Chem., 1999, 7, 2 003-2 012.
30. Oltvoort J. J., van Boeckel C. A. A., Koning J. H., van Boom J. H., Synthesis, 1981, 305-308.

1. Compound capable of binding to gamma-interferon (γ-IFN), chosen from the molecules corresponding to formula (I) below: 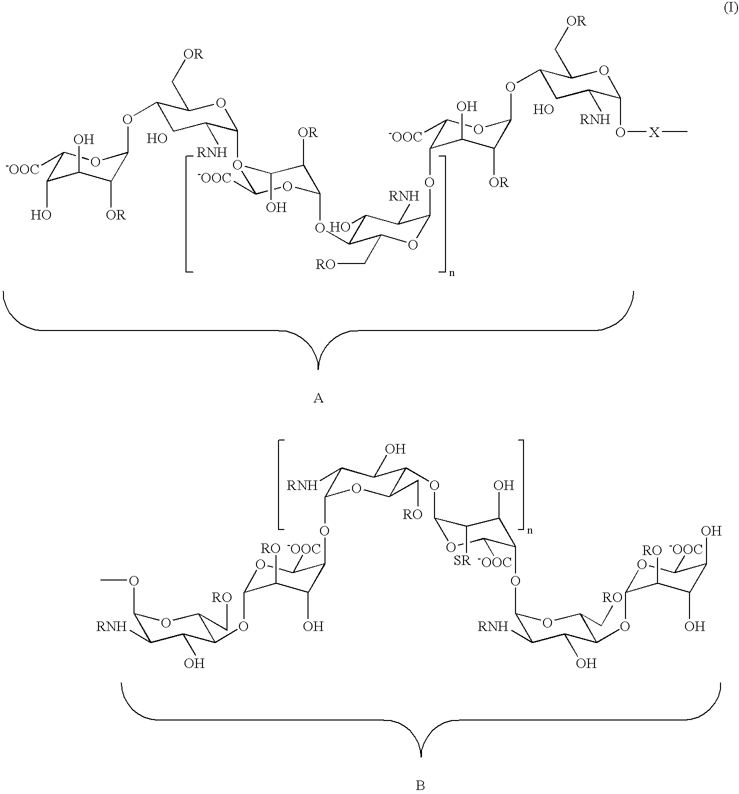
in which X is a divalent spacer group that is sufficiently long to allow the two oligosaccharide fragments A and B to each bind to one of the peptide sequences 125 to 143 of the C-terminal ends of a γ-interferon (γ-IFN) homodimer, n represents an integer from 0 to 10, for example equal to 0, 1, 2, 3, 4 or 5, and each R independently represents a hydrogen atom, an SO₃⁻ group or a phosphate group, with the proviso that no SO₃⁻ group is in the 3-position of the glucosamine units of compound (I).

2. Compound according to claim 1, in which all the R groups represent an SO₃⁻ group or all the R groups represent a phosphate group.

3. Compound according to claim 1, in which the spacer group is 15 to 150 Å, preferably 33 to 50 Å, in length.

4. Compound according to claim 1, in which the spacer
group consists of a carbon chain, preferably of 1 to 120 C, in which one or more of the carbon atoms are optionally replaced with a hetero atom chosen from N, S, P and O, an SO₂ group, or an aryl group, said carbon chain also optionally carrying one or more anionic groups.

5. Compound according to claim 4, in which said anionic groups are chosen from sulphate groups, phosphate groups and carboxylic groups.

6. Compound according to claim 4, in which the spacer group is derived from a polyglycol preferably chosen from poly(alkylene glycols) in which the alkylene group comprises from 1 to 4 C, such as poly(ethylene glycol).

7. Compound according to claim 6, in which the spacer group corresponds to the formula: 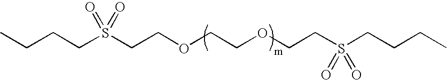
in which m is an integer from 5 to 32.

8. Compound according to claim 7, corresponding to formula (II) below: 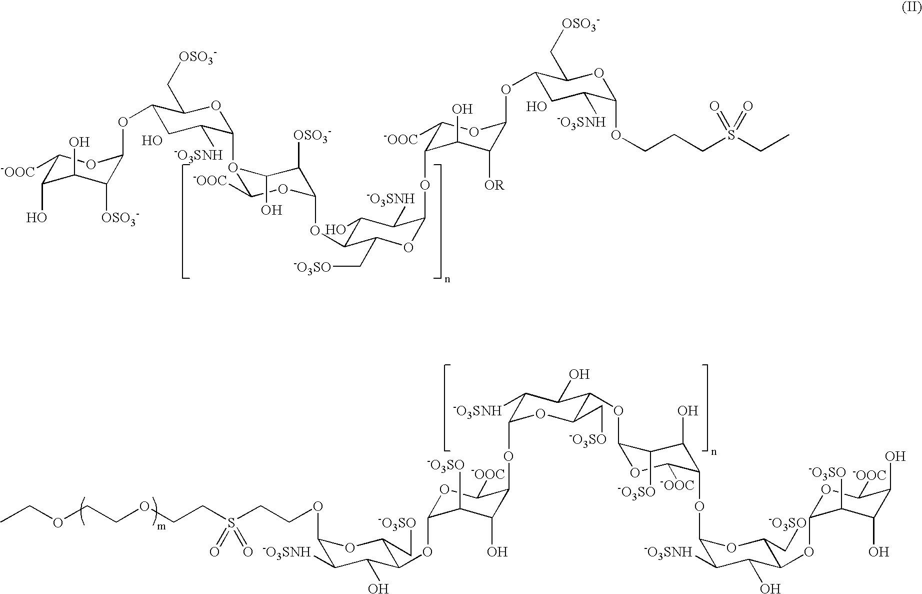
in which n represents an integer from 0 to 10, for example equal to 0, 1, 2, 3, 4 or 5, and m is an integer from 5 to 32.

9. Compound (IIa) corresponding to formula (II) according to claim 8, in which n=0 and m=5.

10. Compound (IIb) corresponding to formula (II) according to claim 8, in which n=0 and m=10.

11. Compound (IIc) corresponding to formula (II) according to claim 8, in which n=0 and m=32.

12. Compound (IId) corresponding to formula (II) according to claim 8, in which n=1 and m=5.

13. Compound (IIe) corresponding to formula (II), according to claim 8, in which n=1 and m=10.

14. Compound (IIf) corresponding to formula (II), according to claim 8, in which n=1 and m=32.

15. Compound (IIg) corresponding to formula (II), according to claim 8, in which n=2 and m=5.

16. Compound (IIh) corresponding to formula (II), according to claim 8, in which n=2 and m=10.

17. Compound (IIi) corresponding to formula (II), according to claim 8, in which n=2 and m=32.

18. Process for preparing a compound capable of binding to gamma-interferon (γ-IFN) of formula (II) according to claim 8, in which the free-radical coupling of two water-soluble compounds that are precursors of oligosaccharides of formula (III): 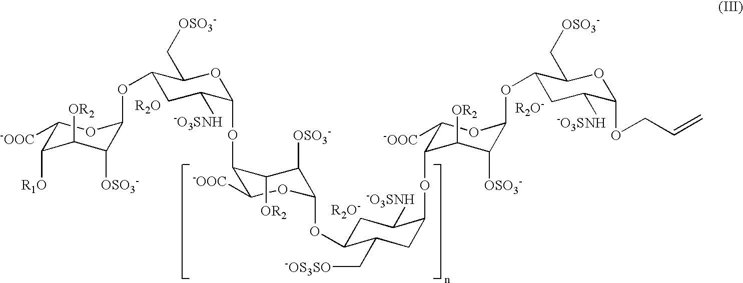
in which n is an integer from 0 to 10, for example equal to 0, 1, 2, 3, 4 or 5, and R₁ and R₂ represent a hydroxyl groulp-protecting group preferably chosen from p-methoxybenzyl and benzyl groups, with a dithiol compound that is a precursor of the spacer group of formula: 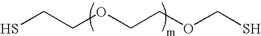
in which m is an integer from 5 to 32, is carried out so as to obtain a compound of formula (IV):
and then, the thioether fluctions are oxidized to sulphones and the final deprotection of compound (IV) is carried out so as to give the final compound of formula (II):

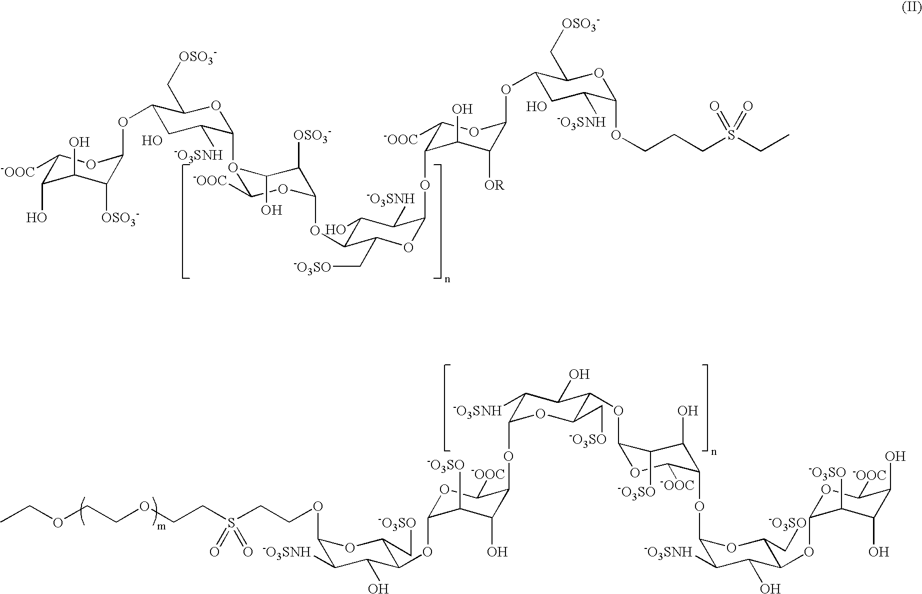

19. Process according to claim 18, in which R₁ is a p-methoxybenzyl group and R₂ is a benzyl group.

20. Process according to claim 18, in which the water-soluble compound that is a precursor of oligosaccharides of formula (III) is prepared by means of the following successive steps:
a) a disaccharide of formula (V): 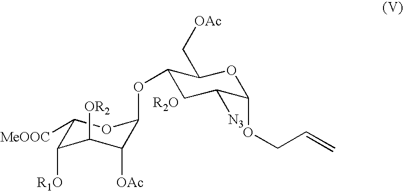
is subjected to oxidative cleavage of the R₁ group, preferably a para-methoxybenzyl group, so as to give an “acceptor” disaccharide of formula: 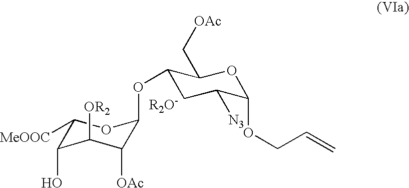
b) in parallel, a disaccharide of formula (V), above, is subjected to isomerization of the allyl group to 1-propenyl, followed by hydrolysis of the enol ether formed and activation of the hydroxyl group in the form of trichloroacetamidate, so as to give a “donor” disaccharide of formula (VIb): 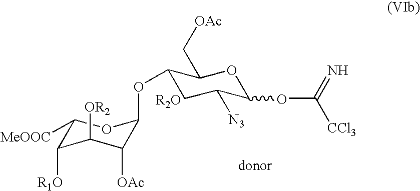
c) the acceptor disaccharide (VIa) and the donor disaccharide (VIb) are coupled so as to obtain the tetrasaccharide (n=0) of formula (VII), with an entirely alpha stereospecificity;
d) optionally, steps a) to c) are repeated, taking the tetrasaccharide prepared in c) as starting product for step a), so as to obtain the hexasaccharide (n=1) and octasaccharide (n=2) of formula (VII): 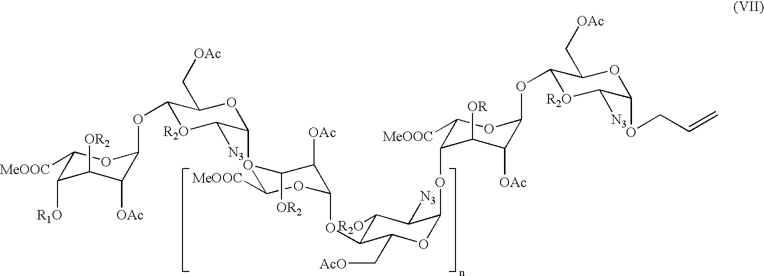
e) optionally, steps a) to c) are repeated, taking the octasaccharide prepared in d) as starting product for step a), so as to obtain a hexadecasaccharide (n=7) of formula (VII);
f) deacetylation, reduction of the azide function, sulphatation and saponification are carried out so as to obtain the desired water-soluble compound that is a precursor of an oligosaccharide (III).

21. Process according to claim 20, in which the disaccharide of formula (V) is prepared by means of a coupling reaction between a compound of formula (VIII): 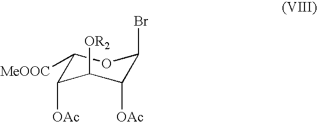
and a compound of formula (IX):

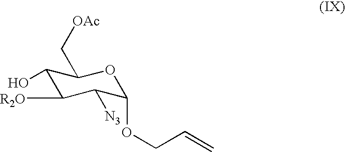

22. Process according to claim 21, in which the compound of formula (IX) is prepared from the compound of formula (X) and the compound of formula (VIII) is prepared from the compound of formula (XI):

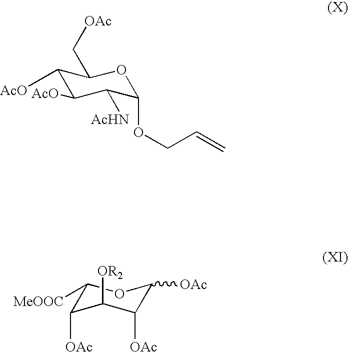

23. Process according to claim 22, in which the compound of formula: 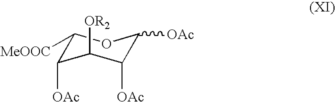
is prepared by acetylation of the compound of formula (XII): 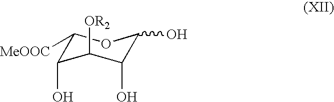
at −40° C. in dichloromethane as solvent, with pyridine as base, acetyl chloride as acylating agent and, 4-dimethylaminopyridine as catalyst.

24. Compound according to claim 1, for use as a medicament.

25. Use of a compound according to claim 1, for preparing a medicament.

26. Compound according to claim 1, for use as a modulator, for example inhibitor, of the activity of endogenous or exogenous γ-interferon.

27. Compound according to claim 1, for use in the treatment of diseases associated with, or characterized by, the presence of pro-inflammatory cytokines such as γ-interferon, for example autoimmune, inflammatory or degenerative diseases such as multiple sclerosis, glomerulonephritis, Crohn's disease and rheumatoid arthritis.

28. Compound according to claim 1, for use in a treatment to supplement the immuno-suppressive treatments used, for example, for preventing transplant rejection.

29. Medicament containing a compound according to claim 1.

30. Composition containing the compound according to claim 1 and a pharmaceutically acceptable vehicle, for use in the treatment of diseases associated with, or characterized by, the presence of pro-inflammatory cytokines such as γ-interferon, for example autoimmune or degenerative diseases such as multiple sclerosis, glomerulonephritis, Crohn's disease and rheumatoid arthritis.

31. Composition containing the compound according to claim 1 and a pharmaceutically acceptable vehicle, for use in a treatment to supplement the immunosuppressive treatments used, for example, to prevent transplant rejection.

32. Use of a compound according to claim 1, for preparing a medicament intended for the treatment of pathologies or conditions related to the activity, in particular excessive activity, of endogenous or exogenous γ-interferon.

33. Use of a compound according to claim 1, for preparing a medicament intended for the treatment of diseases associated with, or characterized by, the presence of pro-inflammatory cytokines such as γ-interferon, for example autoimmune, inflammatory or degenerative diseases such as multiple sclerosis, glomerulonephritis, Crohn's disease and rheumatoid arthritis.

34. Use of a compound according to claim 1, for preparing a medicament intended for a treatment to supplement the immunosuppressive treatments used, for example, for preventing transplant rejection.

35. Medicament containing γ-interferon in addition to a compound according to claim 1.

36. Medicament according to claim 35, in which the compound according to claim 1 and the γ-interferon are in the form of a complex of the compound and of the γ-interferon.

37. Complex of a compound according to claim 1 and of γ-interferon, for use as a medicament.

38. Complex of a compound according to claim 1 and of γ-interferon, for use as an immunostimulant.

39. Complex of a compound according to claim 1 and of γ-interferon, for use in the treatment of a disease chosen from cancer, infectious, for example viral, bacterial or parasitic, diseases, and organ fibroses.

40. Composition containing a complex of a compound according to claim 1 and of γ-interferon, and a pharmaceutically acceptable vehicle, for use in the treatment of a disease chosen from cancer, infectious, for example viral, bacterial or parasitic, diseases, and organ fibroses.

41. Medicament containing a complex of a compound according to claim 1 and of γ-interferon.

42. Use of a complex of a compound according to claim 1 and of γ-interferon, for preparing a medicament.

43. se of a complex of a compound according to claim 1 and of γ-interferon, for preparing a medicament intended for the treatment of a given disease among cancer, infectious, for example, viral, bacterial or parasitic, diseases, and organ fibrosis.
